SEQUENCE LISTING (4 sequences)

SEQ ID NO: 1 - protein, 8 aa, synthetic construct
LMSTNDLK

SEQ ID NO: 2 - protein, 15 aa, synthetic construct
LDLTPSPEVPLTILK

SEQ ID NO: 3 - protein, 438 aa, Cricetulus sp.
MCRMIWKAAVLLSLALGAGALAVGVDDPEDAGKHWVVIVAGSNGWYNYRHQADACHAYQIIHRNGIPDEQIIVMMYDDIANSEDNPTPGIVINRPNGTDVYAGVLKDYTGEDVTPENFLAVLRGDAEAVKGKGSGKVLRSGPQDHVFVYFTDHGATGLLVFPNEDLHVKDLNKTIRYMYEHKMYQKMVFYIEACESGSMMNHLPNDINVYATTAANPHESSYACYYDEERNTYLGDWYSVNWMEDSDVEDLTKETLHKQYHLVKSHTNTSHVMQYGNKSISTMKVMQFQGMKHSTSSPISLPPVTRLDLTPSPEVPLTILKRKLMSTNDLKQSQNLVGQIQRLLDARHVIEKSVHKIVSLLAGFGETAERLLSERAVLMAHDCYQEAVTHFRTHCFNWHSPTYEYALRHLYVLANLCEKPYPIDRIKMAMDKVCLSHY

SEQ ID NO: 4 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

(19) United States
(12) Patent Application Publication
(10) Pub. No.: US 2024/0262908 A1
(43) Pub. Date: Aug. 8, 2024

(54) MITIGATION OF THERAPEUTIC PROTEIN FRAGMENTATION DURING CELL CULTURE HARVEST PROCESSES

(71) Applicant: AMGEN INC., Thousand Oaks, CA (US)

(72) Inventors: Gang Xiao, Thousand Oaks, CA (US); Hyo Seon Chung, La Canada Flintridge, CA (US); Quanzhou Luo, Oak Park, CA (US); Tamer Eris, Ventura, CA (US); Joseph Edward Basconi, Cambridge, MA (US); Xiaoyang Zhao, Moorpark, CA (US); Pavel Bondarenko, Thousand Oaks, CA (US); Krista Marie Petty, Ventura, CA (US); Oliver Kaltenbrunner, Thousand Oaks, CA (US)

(73) Assignee: AMGEN INC., Thousand Oaks, CA (US)

(21) Appl. No.: 18/429,717

(22) Filed: Feb. 1, 2024

Related U.S. Application Data
(60) Provisional application No. 63/482,844, filed on Feb. 2, 2023.

(51) Int. Cl. C07K 16/28; C12N 5/071; C12N 9/64

(52) U.S. Cl. CPC C07K 16/2803; C12N 5/0682; C12N 9/641; C07K 2317/522; C12N 2500/02; C12N 2501/999; C12N 2523/00; C12Y 304/22034

(57) ABSTRACT

Disclosed herein are methods for harvesting a recombinant protein comprising an asparaginyl endopeptidase (AEP) cleavage site, as well as methods for inhibiting cleavage of a recombinant protein comprising an AEP cleavage site, during a biomanufacturing process.

### CROSS-REFERENCE TO RELATED APPLICATIONS

This application claims the benefit of priority to U.S. Provisional Patent Application No. 63/482,844, filed Feb. 2, 2023, which is hereby incorporated by reference in its entirety for all purposes.

### SUBMISSION OF SEQUENCE LISTING

The instant application contains a computer-readable Sequence Listing which has been submitted electronically in XML format and is hereby incorporated by reference in its entirety. Said XML copy, created on Jan. 25, 2024, is named “10177-US02-SEC_ST26” and is 5,111 bytes in size.

### FIELD

The present disclosure relates to methods for harvesting a recombinant protein comprising an asparaginyl endopeptidase (AEP) cleavage site, wherein the methods employ an acid precipitation operation at a pH of about 4.6 to about 5.3 and optionally: (i) employ a dissolved oxygen level of about 64 mmHg to about 128 mmHg during cell culture cooling and/or harvesting; and/or (ii) utilize an exogenous protease inhibitor, e.g., during one or more of a cell culture growth phase, a cell culture production phase, and/or a cell culture cooling. The present disclosure also relates to methods for inhibiting cleavage of a recombinant protein comprising an AEP cleavage site during a biomanufacturing process comprising an acid precipitation operation, wherein the acid precipitation pH is adjusted to about 4.6 to about 5.3 if one or more parameters (such as, e.g., fragment quantity; AEP quantity or activity) exceed a threshold. Furthermore, the present disclosure relates to methods for monitoring activation of AEP in a sample from a biomanufacturing process utilizing Chinese Hamster Ovary (CHO) cells, wherein the methods comprise subjecting the sample to trypsin to obtain a trypsin-digest sample and measuring the amount of LMSTNDLK (SEQ ID NO: 1) and/or LDLTPSPEVPLTILK (SEQ ID NO: 2) in the trypsin-digest sample.

### BACKGROUND

In response to strong and growing demand for biological therapeutics, significant advances in bioprocessing have been made to facilitate cost-efficient, large-scale recombinant protein production. Illustratively, new and improved high cell density culture methods and intensified cell culture processes have enabled greater volumetric productivity at reduced cost. However, in addition to increasing protein titers, these upstream processes typically result in higher cell densities and process-related impurity levels (e.g., host cell proteins (HCPs) and nucleic acids), which increase the burden on the costly downstream clarification and purification operations used to isolate recombinant proteins.

Cell culture clarification is a downstream unit operation in which cells, cellular debris, and other process-related impurities are removed from cell culture harvest fluid prior to further downstream purification steps, such as, e.g., chromatographic separation processes. Mechanical separation (such as, e.g., centrifugation) followed by depth filtration is a common approach for clarifying cell cultures. To increase filtration throughput and improve separation performance, many clarification processes incorporate a pretreatment step, such as acid precipitation or flocculant addition, that renders certain process-related impurities insoluble prior to mechanical separation of the remaining cell culture fluid (CCF).

However, as with all downstream unit operations, the process conditions used during cell harvesting must be carefully controlled to avoid product loss and product degradation. This is especially true when processing large scale cell biomasses due to the high concentration of lysosomal enzymes, such as cathepsins and pepsins, that may be secreted or released into the bioreactor during cell culture and harvest. Although lysosomal enzymes are not active under the neutral pH conditions employed during cell culture, these enzymes may become active under acidic and mildly acidic pH conditions, such as those used during cell culture harvesting and protein purification.

Accordingly, there is a need in the art for new and improved cell culture harvesting methods that enable efficient clarification of high cell density cultures without inducing product cleavage by lysosomal enzymes that may be present within the cell culture.

### SUMMARY

Disclosed herein is a method for harvesting a recombinant protein during a biomanufacturing process comprising:
admixing an acidic solution with a cell culture comprising the recombinant protein to obtain an acidified cell culture with a pH of about 4.6 to about 5.3; and
incubating the acidified cell culture to induce agglomeration of one or more cell culture impurities,
wherein the recombinant protein comprises an asparaginyl endopeptidase (AEP) cleavage site.

In some embodiments, an exogenous protease inhibitor is admixed with the cell culture before admixing with the acidic solution. In some embodiments, an exogenous protease inhibitor is admixed with the acidified cell culture. In some embodiments, the exogenous protease inhibitor is an AEP inhibitor.

In some embodiments, an exogenous protease inhibitor is added to a pool downstream of the acidified cell culture (such as, e.g., a protein A pool or a low pH virus inactivation pool). In some embodiments, the exogenous protease inhibitor is an AEP inhibitor.

In some embodiments, the method comprises incubating the acidified cell culture at a temperature of about 4° C. to about 37° C. In some embodiments, the method comprises incubating the acidified cell culture at a temperature of about 15° C. to about 20° C.

In some embodiments, the acidic solution comprises an acid selected from acetic acid, trichloroacetic acid, formic acid, phosphoric acid, sulfuric acid, citric acid, caprylic acid, and combinations of any of the foregoing. In some embodiments, the acidic solution comprises an acid selected from acetic acid, phosphoric acid, and combinations thereof. In some embodiments, the acidic solution comprises sulfuric acid. In some embodiments, the acidic solution comprises citric acid. In some embodiments, the acidic solution comprises acetic acid. In some embodiments, the acidic solution comprises 1 M acetic acid. In some embodiments, the acidic solution comprises phosphoric acid. In some embodiments, the acidic solution comprises 2 M phosphoric acid.

In some embodiments, the method comprises incubating the acidified cell culture for at least about 30 minutes. In some embodiments, the method comprises incubating the acidified cell culture for at least about 40 minutes. In some embodiments, the method comprises incubating the acidified cell culture for at least about 45 minutes. In some embodiments, the method comprises incubating the acidified cell culture for at least about 60 minutes.

In some embodiments, the method comprises incubating the acidified cell culture for about 5 minutes to about 48 hours. In some embodiments, the method comprises incubating the acidified cell culture for about 5 minutes to about 24 hours. In some embodiments, the method comprises incubating the acidified cell culture for about 5 minutes to about 12 hours. In some embodiments, the method comprises incubating the acidified cell culture for about 5 minutes to about 120 minutes. In some embodiments, the method comprises incubating the acidified cell culture for about 5 minutes to about 90 minutes. In some embodiments, the method comprises incubating the acidified cell culture for about 5 minutes to about 60 minutes. In some embodiments, the method comprises incubating the acidified cell culture for about 30 minutes to about 90 minutes.

In some embodiments, the method further comprises separating the one or more cell culture impurities from the acidified cell culture to obtain a clarified cell culture. In some embodiments, the one or more cell culture impurities are separated from the acidified cell culture by centrifugation and/or depth filtration. In some embodiments, the one or more cell culture impurities are separated from the acidified cell culture by centrifugation. In some embodiments, the one or more cell culture impurities are separated from the acidified cell culture by continuous solid discharge centrifugation, disk stack centrifugation, and/or depth filtration. In some embodiments, the one or more cell culture impurities are separated from the acidified cell culture by continuous solid discharge centrifugation. In some embodiments, the one or more cell culture impurities are separated from the acidified cell culture by disk stack centrifugation. In some embodiments, the one or more cell culture impurities are separated from the acidified cell culture by intermittent discharge centrifugation. In some embodiments, the one or more cell culture impurities are separated from the acidified cell culture by depth filtration. In some embodiments, the one or more cell culture impurities are separated from the acidified cell culture by depth filtration without centrifugation. In some embodiments, the one or more cell culture impurities are separated from the acidified cell culture by continuous solid discharge centrifugation and depth filtration.

In some embodiments, the depth filtration is performed at a loading of less than about 150 L/m². In some embodiments, the depth filtration is performed at a loading of less than about 140 L/m². In some embodiments, the depth filtration is performed at a loading of less than about 130 L/m². In some embodiments, the depth filtration is performed at a loading of less than about 120 L/m². In some embodiments, the depth filtration is performed at a loading of less than about 110 L/m². In some embodiments, the depth filtration is performed at a loading of less than about 100 L/m². In some embodiments, the depth filtration is performed at a loading of less than about 95 L/m². In some embodiments, the depth filtration is performed at a loading of less than about 90 L/m². In some embodiments, the depth filtration is performed at a loading of less than about 85 L/m². In some embodiments, the depth filtration is performed at a loading of less than about 80 L/m². In some embodiments, the depth filtration is performed at a loading of less than about 75 L/m².

In some embodiments, the method comprises neutralizing the acidified cell culture prior to separating the one or more cell culture impurities from the acidified cell culture. In some embodiments, the method does not comprise neutralizing the acidified cell culture prior to separating the one or more cell culture impurities from the acidified cell culture.

In some embodiments, the method comprises neutralizing the clarified cell culture.

In some embodiments, the one or more cell culture impurities comprise cell culture debris, host cell proteins, and nucleic acids. In some embodiments, the one or more cell culture impurities comprise cell culture debris. In some embodiments, the one or more cell culture impurities comprise host cell proteins. In some embodiments, the one or more cell culture impurities comprise nucleic acids. In some embodiments, the one or more cell culture impurities are selected from host cell proteins and nucleic acids.

In some embodiments, the pH of the acidified cell culture is about 4.7 to about 5.3. In some embodiments, the pH of the acidified cell culture is about 4.8 to about 5.3. In some embodiments, the pH of the acidified cell culture is about 4.9 to about 5.3. In some embodiments, the pH of the acidified cell culture is about 5.0 to about 5.3. In some embodiments, the pH of the acidified cell culture is about 4.7 to about 5.2. In some embodiments, the pH of the acidified cell culture is about 4.8 to about 5.2. In some embodiments, the pH of the acidified cell culture is about 4.9 to about 5.2. In some embodiments, the pH of the acidified cell culture is about 5.0 to about 5.2.

In some embodiments, the pH of the acidified cell culture is about 4.6. In some embodiments, the pH of the acidified cell culture is about 4.7. In some embodiments, the pH of the acidified cell culture is about 4.8. In some embodiments, the pH of the acidified cell culture is about 4.9. In some embodiments, the pH of the acidified cell culture is about 5.0. In some embodiments, the pH of the acidified cell culture is about 5.1. In some embodiments, the pH of the acidified cell culture is about 5.2. In some embodiments, the pH of the acidified cell culture is about 5.3.

In some embodiments, the pH of the acidified cell culture deviates by less than about ±0.2 pH units during the incubation. In some embodiments, the pH of the acidified cell culture deviates by less than about ±0.1 pH units during the incubation.

In some embodiments, the pH of the acidified cell culture deviates by less than about 25% during the incubation. In some embodiments, the pH of the acidified cell culture deviates by less than about 20% during the incubation. In some embodiments, the pH of the acidified cell culture deviates by less than about 15% during the incubation. In some embodiments, the pH of the acidified cell culture deviates by less than about 10% during the incubation. In some embodiments, the pH of the acidified cell culture deviates by less than about 5% during the incubation.

In some embodiments, the acidified cell culture comprises recombinant protein comprising a sequence upstream of the AEP cleavage site and a sequence downstream of the AEP cleavage site. In some embodiments, the acidified cell culture comprises recombinant protein comprising an antibody heavy chain sequence upstream of the AEP cleavage site and an antibody heavy chain sequence downstream of the AEP cleavage site.

In some embodiments, the acidified cell culture comprises a reduced quantity of one or more fragments of the recombinant protein relative to an alternative acidified cell culture with a pH of less than about 4.6. In some embodiments, the fragments of the recombinant protein comprise species cleaved at the AEP cleavage site.

In some embodiments, less than about 5% w/w of the recombinant protein in the acidified cell culture is fragments of the recombinant protein. In some embodiments, less than about 4% w/w of the recombinant protein in the acidified cell culture is fragments of the recombinant protein. In some embodiments, less than about 3% w/w of the recombinant protein in the acidified cell culture is fragments of the recombinant protein. In some embodiments, less than about 2% w/w of the recombinant protein in the acidified cell culture is fragments of the recombinant protein. In some embodiments, less than about 1% w/w of the recombinant protein in the acidified cell culture is fragments of the recombinant protein. Fragment content may be assessed, e.g., via UV absorbance relative peak area on reduced capillary electrophoresis.

In some embodiments, the method further comprises measuring AEP quantity or activity prior to the admixing. In some embodiments, the method further comprises measuring AEP quantity prior to the admixing. In some embodiments, the method further comprises measuring AEP activity prior to the admixing. In some embodiments, the method further comprises measuring AEP proenzyme quantity prior to the admixing.

In some embodiments, the AEP or AEP proenzyme quantity is measured using mass spectrometry or an immunoassay. In some embodiments, the AEP or AEP proenzyme quantity is measured using mass spectrometry. In some embodiments, the AEP or AEP proenzyme quantity is measured using an immunoassay.

In some embodiments, the biomanufacturing process utilizes CHO cells and the AEP proenzyme quantity is measured by subjecting a pool sample to trypsin to obtain a trypsin-digest sample and measuring the amount of LMSTNDLK (SEQ ID NO: 1) and/or LDLTPSPEVPLTILK (SEQ ID NO: 2) in the trypsin-digest sample. In some embodiments, the method further comprises comparing the measured amount of SEQ ID NO: 1 and/or SEQ ID NO: 2 to one or more benchmark values, such as, e.g., a measured amount in a trypsin-digest sample comprising a known amount of AEP at a pH greater than or equal to 5.5.

In some embodiments, the AEP activity is measured using a cleavage assay.

In some embodiments, the cell culture comprises mammalian cells. In some embodiments, the cell culture comprises Chinese Hamster Ovary (CHO) cells.

In some embodiments, the recombinant protein is an antigen-binding protein. In some embodiments, the recombinant protein is an antibody. In some embodiments, the recombinant protein is an IgG2 antibody. In some embodiments, the recombinant protein comprises an IgG2 heavy chain constant region. In some embodiments, the recombinant protein comprises a wild-type IgG2 heavy chain constant region. In some embodiments, the recombinant protein comprises an engineered IgG2 heavy chain constant region.

In some embodiments, the recombinant protein comprises a N192/F193 sequence motif in a CH1 domain of a heavy chain constant region, according to EU numbering.

In some embodiments, the biomanufacturing process comprises a perfusion cell culture process, a concentrated fed batch cell culture process, or an intensified cell culture process. In some embodiments, the biomanufacturing process comprises a perfusion cell culture process. In some embodiments, the biomanufacturing process comprises a concentrated fed batch cell culture process. In some embodiments, the biomanufacturing process comprises an intensified cell culture process.

In some embodiments, the biomanufacturing process comprises a production phase with a viable cell density of at least about 10⁶ cells/mL. In some embodiments, the biomanufacturing process comprises a production phase with a viable cell density of at least about 5×10⁶ cells/mL. In some embodiments, the biomanufacturing process comprises a production phase with a viable cell density of at least about 10⁷ cells/mL.

In some embodiments, the biomanufacturing process comprises a production phase with a packed cell volume of about 2% to about 40%. In some embodiments, the biomanufacturing process comprises a production phase with a packed cell volume of about 15% to about 18%. In some embodiments, the biomanufacturing process comprises a production phase with a packed cell volume of about 25% to about 35%. In some embodiments, the biomanufacturing process comprises a production phase with a packed cell volume of about 3% to about 15%.

In some embodiments, the biomanufacturing process comprises a production phase performed in a bioreactor with a capacity of at least about 500 L. In some embodiments, the biomanufacturing process comprises a production phase performed in a bioreactor with a capacity of at least about 1 kL. In some embodiments, the biomanufacturing process comprises a production phase performed in a bioreactor with a capacity of at least about 2 kL. In some embodiments, the biomanufacturing process comprises a production phase performed in a bioreactor with a capacity of at least about 5 kL. In some embodiments, the biomanufacturing process comprises a production phase performed in a bioreactor with a capacity of at least about 10 kL. In some embodiments, the biomanufacturing process comprises a production phase performed in a bioreactor with a capacity of at least about 20 kL.

In some embodiments, the biomanufacturing process comprises a production phase performed in a bioreactor with a capacity of about 500 L. In some embodiments, the biomanufacturing process comprises a production phase performed in a bioreactor with a capacity of about 1 kL. In some embodiments, the biomanufacturing process comprises a production phase performed in a bioreactor with a capacity of about 2 kL. In some embodiments, the biomanufacturing process comprises a production phase performed in a bioreactor with a capacity of about 5 kL. In some embodiments, the biomanufacturing process comprises a production phase performed in a bioreactor with a capacity of about 10 kL. In some embodiments, the biomanufacturing process comprises a production phase performed in a bioreactor with a capacity of about 20 kL.

Also disclosed herein is a method for harvesting a recombinant protein during a biomanufacturing process comprising:
establishing a cell culture by inoculating a bioreactor with mammalian cells expressing the recombinant protein;
maintaining the cell culture during a growth phase and a production phase;
cooling the cell culture, wherein a dissolved oxygen level of about 64 mmHg to about 128 mmHg is maintained in the cell culture during the cooling;
admixing an acidic solution with the cell culture to obtain an acidified cell culture with a pH of about 4.6 to about 5.3; and
incubating the acidified cell culture to induce agglomeration of one or more cell culture impurities,
wherein the recombinant protein comprises an asparaginyl endopeptidase (AEP) cleavage site.

In some embodiments, the cell culture is cooled to about 4° C. to about 15° C. In some embodiments, the cell culture is cooled to about 4° C. to about 10° C. In some embodiments, the cell culture is cooled to about 9° C. to about 11° C. In some embodiments, the cell culture is cooled to about 10° C.

In some embodiments, the method comprises incubating the acidified cell culture at a temperature of about 4° C. to about 37° C. In some embodiments, the method comprises incubating the acidified cell culture at a temperature of about 15° C. to about 20° C.

In some embodiments, an exogenous protease inhibitor is admixed with the cell culture during the growth phase, the production phase, and/or the cooling phase. In some embodiments, an exogenous protease inhibitor is admixed with the cell culture after the production phase. In some embodiments, an exogenous protease inhibitor is admixed with the cell culture after the production phase and before the cooling phase.

In some embodiments, an exogenous protease inhibitor is admixed with the cell culture before admixing with the acidic solution. In some embodiments, an exogenous protease inhibitor is admixed with the acidified cell culture. In some embodiments, the exogenous protease inhibitor is an AEP inhibitor.

In some embodiments, an exogenous protease inhibitor is added to a pool downstream of the acidified cell culture (such as, e.g., a protein A pool or a low pH virus inactivation pool). In some embodiments, the exogenous protease inhibitor is an AEP inhibitor.

In some embodiments, the acidic solution comprises an acid selected from acetic acid, trichloroacetic acid, formic acid, phosphoric acid, sulfuric acid, citric acid, caprylic acid, and combinations of any of the foregoing. In some embodiments, the acidic solution comprises an acid selected from acetic acid, phosphoric acid, and combinations thereof. In some embodiments, the acidic solution comprises sulfuric acid. In some embodiments, the acidic solution comprises citric acid. In some embodiments, the acidic solution comprises acetic acid. In some embodiments, the acidic solution comprises 1 M acetic acid. In some embodiments, the acidic solution comprises phosphoric acid. In some embodiments, the acidic solution comprises 2 M phosphoric acid.

In some embodiments, the method comprises incubating the acidified cell culture for at least about 30 minutes. In some embodiments, the method comprises incubating the acidified cell culture for at least about 40 minutes. In some embodiments, the method comprises incubating the acidified cell culture for at least about 45 minutes. In some embodiments, the method comprises incubating the acidified cell culture for at least about 60 minutes.

In some embodiments, the method comprises incubating the acidified cell culture for about 5 minutes to about 48 hours. In some embodiments, the method comprises incubating the acidified cell culture for about 5 minutes to about 24 hours. In some embodiments, the method comprises incubating the acidified cell culture for about 5 minutes to about 12 hours. In some embodiments, the method comprises incubating the acidified cell culture for about 5 minutes to about 120 minutes. In some embodiments, the method comprises incubating the acidified cell culture for about 5 minutes to about 90 minutes. In some embodiments, the method comprises incubating the acidified cell culture for about 5 minutes to about 60 minutes. In some embodiments, the method comprises incubating the acidified cell culture for about 30 minutes to about 90 minutes.

In some embodiments, the method further comprises separating the one or more cell culture impurities from the acidified cell culture to obtain a clarified cell culture. In some embodiments, the one or more cell culture impurities are separated from the acidified cell culture by centrifugation and/or depth filtration. In some embodiments, the one or more cell culture impurities are separated from the acidified cell culture by centrifugation. In some embodiments, the one or more cell culture impurities are separated from the acidified cell culture by continuous solid discharge centrifugation, disk stack centrifugation, and/or depth filtration. In some embodiments, the one or more cell culture impurities are separated from the acidified cell culture by continuous solid discharge centrifugation. In some embodiments, the one or more cell culture impurities are separated from the acidified cell culture by disk stack centrifugation. In some embodiments, the one or more cell culture impurities are separated from the acidified cell culture by intermittent discharge centrifugation. In some embodiments, the one or more cell culture impurities are separated from the acidified cell culture by depth filtration. In some embodiments, the one or more cell culture impurities are separated from the acidified cell culture by depth filtration without centrifugation. In some embodiments, the one or more cell culture impurities are separated from the acidified cell culture by continuous solid discharge centrifugation and depth filtration.

In some embodiments, the depth filtration is performed at a loading of less than about 150 L/m². In some embodiments, the depth filtration is performed at a loading of less than about 140 L/m². In some embodiments, the depth filtration is performed at a loading of less than about 130 L/m². In some embodiments, the depth filtration is performed at a loading of less than about 120 L/m². In some embodiments, the depth filtration is performed at a loading of less than about 110 L/m². In some embodiments, the depth filtration is performed at a loading of less than about 100 L/m². In some embodiments, the depth filtration is performed at a loading of less than about 95 L/m². In some embodiments, the depth filtration is performed at a loading of less than about 90 L/m². In some embodiments, the depth filtration is performed at a loading of less than about 85 L/m². In some embodiments, the depth filtration is performed at a loading of less than about 80 L/m². In some embodiments, the depth filtration is performed at a loading of less than about 75 L/m².

In some embodiments, the method comprises neutralizing the acidified cell culture prior to separating the one or more cell culture impurities from the acidified cell culture. In some embodiments, the method does not comprise neutralizing the acidified cell culture prior to separating the one or more cell culture impurities from the acidified cell culture.

In some embodiments, the method comprises neutralizing the clarified cell culture.

In some embodiments, the one or more cell culture impurities comprise cell culture debris, host cell proteins, and nucleic acids. In some embodiments, the one or more cell culture impurities comprise cell culture debris. In some embodiments, the one or more cell culture impurities comprise host cell proteins. In some embodiments, the one or more cell culture impurities comprise nucleic acids. In some embodiments, the one or more cell culture impurities are selected from host cell proteins and nucleic acids.

In some embodiments, the pH of the acidified cell culture is about 4.7 to about 5.3. In some embodiments, the pH of the acidified cell culture is about 4.8 to about 5.3. In some embodiments, the pH of the acidified cell culture is about 4.9 to about 5.3. In some embodiments, the pH of the acidified cell culture is about 5.0 to about 5.3. In some embodiments, the pH of the acidified cell culture is about 4.7 to about 5.2. In some embodiments, the pH of the acidified cell culture is about 4.8 to about 5.2. In some embodiments, the pH of the acidified cell culture is about 4.9 to about 5.2. In some embodiments, the pH of the acidified cell culture is about 5.0 to about 5.2.

In some embodiments, the pH of the acidified cell culture is about 4.6. In some embodiments, the pH of the acidified cell culture is about 4.7. In some embodiments, the pH of the acidified cell culture is about 4.8. In some embodiments, the pH of the acidified cell culture is about 4.9. In some embodiments, the pH of the acidified cell culture is about 5.0. In some embodiments, the pH of the acidified cell culture is about 5.1. In some embodiments, the pH of the acidified cell culture is about 5.2. In some embodiments, the pH of the acidified cell culture is about 5.3.

In some embodiments, the pH of the acidified cell culture deviates by less than about ±0.2 pH units during the incubation. In some embodiments, the pH of the acidified cell culture deviates by less than about ±0.1 pH units during the incubation.

In some embodiments, the pH of the acidified cell culture deviates by less than about 25% during the incubation. In some embodiments, the pH of the acidified cell culture deviates by less than about 20% during the incubation. In some embodiments, the pH of the acidified cell culture deviates by less than about 15% during the incubation. In some embodiments, the pH of the acidified cell culture deviates by less than about 10% during the incubation. In some embodiments, the pH of the acidified cell culture deviates by less than about 5% during the incubation.

In some embodiments, the acidified cell culture comprises recombinant protein comprising a sequence upstream of the AEP cleavage site and a sequence downstream of the AEP cleavage site.

In some embodiments, the acidified cell culture comprises a reduced quantity of one or more fragments of the recombinant protein relative to an alternative acidified cell culture with a pH of less than about 4.6. In some embodiments, the fragments of the recombinant protein comprise species cleaved at the AEP cleavage site.

In some embodiments, less than about 5% w/w of the recombinant protein in the acidified cell culture is fragments of the recombinant protein. In some embodiments, less than about 4% w/w of the recombinant protein in the acidified cell culture is fragments of the recombinant protein. In some embodiments, less than about 3% w/w of the recombinant protein in the acidified cell culture is fragments of the recombinant protein. In some embodiments, less than about 2% w/w of the recombinant protein in the acidified cell culture is fragments of the recombinant protein. In some embodiments, less than about 1% w/w of the recombinant protein in the acidified cell culture is fragments of the recombinant protein. Fragment content may be assessed, e.g., via UV absorbance relative peak area on reduced capillary electrophoresis.

In some embodiments, the method further comprises measuring AEP quantity or activity prior to the admixing. In some embodiments, the method further comprises measuring AEP quantity prior to the admixing. In some embodiments, the method further comprises measuring AEP activity prior to the admixing. In some embodiments, the method further comprises measuring AEP proenzyme quantity prior to the admixing.

In some embodiments, the AEP or AEP proenzyme quantity is measured using mass spectrometry or an immunoassay. In some embodiments, the AEP or AEP proenzyme quantity is measured using mass spectrometry. In some embodiments, the AEP or AEP proenzyme quantity is measured using an immunoassay.

In some embodiments, the biomanufacturing process utilizes CHO cells and the AEP proenzyme quantity is measured by subjecting a pool sample to trypsin to obtain a trypsin-digest sample and measuring the amount of LMSTNDLK (SEQ ID NO: 1) and/or LDLTPSPEVPLTILK (SEQ ID NO: 2) in the trypsin-digest sample. In some embodiments, the method further comprises comparing the measured amount of SEQ ID NO: 1 and/or SEQ ID NO: 2 to one or more benchmark values, such as, e.g., a measured amount in a trypsin-digest sample comprising a known amount of AEP at a pH greater than or equal to 5.5.

In some embodiments, the AEP activity is measured using a cleavage assay.

In some embodiments, the mammalian cells are CHO cells.

In some embodiments, the recombinant protein is an antigen-binding protein. In some embodiments, the recombinant protein is an antibody. In some embodiments, the recombinant protein is an IgG2 antibody. In some embodiments, the recombinant protein comprises an IgG2 heavy chain constant region. In some embodiments, the recombinant protein comprises a wild-type IgG2 heavy chain constant region. In some embodiments, the recombinant protein comprises an engineered IgG2 heavy chain constant region.

In some embodiments, the recombinant protein comprises a N192/F193 sequence motif in a CH1 domain of a heavy chain constant region, according to EU numbering.

In some embodiments, the biomanufacturing process comprises a perfusion cell culture process, a concentrated fed batch cell culture process, or an intensified cell culture process. In some embodiments, the biomanufacturing process comprises a perfusion cell culture process. In some embodiments, the biomanufacturing process comprises a concentrated fed batch cell culture process. In some embodiments, the biomanufacturing process comprises an intensified cell culture process.

In some embodiments, the biomanufacturing process comprises a production phase with a viable cell density of at least about 10⁶ cells/mL. In some embodiments, the biomanufacturing process comprises a production phase with a viable cell density of at least about 5×10⁶ cells/mL. In some embodiments, the biomanufacturing process comprises a production phase with a viable cell density of at least about 10⁷ cells/mL.

In some embodiments, the biomanufacturing process comprises a production phase with a packed cell volume of about 2% to about 40%. In some embodiments, the biomanufacturing process comprises a production phase with a packed cell volume of about 15% to about 18%. In some embodiments, the biomanufacturing process comprises a production phase with a packed cell volume of about 25% to about 35%. In some embodiments, the biomanufacturing process comprises a production phase with a packed cell volume of about 3% to about 15%.

In some embodiments, the biomanufacturing process comprises a production phase performed in a bioreactor with a capacity of at least about 500 L. In some embodiments, the biomanufacturing process comprises a production phase performed in a bioreactor with a capacity of at least about 1 kL. In some embodiments, the biomanufacturing process comprises a production phase performed in a bioreactor with a capacity of at least about 2 kL. In some embodiments, the biomanufacturing process comprises a production phase performed in a bioreactor with a capacity of at least about 5 kL. In some embodiments, the biomanufacturing process comprises a production phase performed in a bioreactor with a capacity of at least about 10 KL. In some embodiments, the biomanufacturing process comprises a production phase performed in a bioreactor with a capacity of at least about 20 kL.

In some embodiments, the biomanufacturing process comprises a production phase performed in a bioreactor with a capacity of about 500 L. In some embodiments, the biomanufacturing process comprises a production phase performed in a bioreactor with a capacity of about 1 kL. In some embodiments, the biomanufacturing process comprises a production phase performed in a bioreactor with a capacity of about 2 kL. In some embodiments, the biomanufacturing process comprises a production phase performed in a bioreactor with a capacity of about 5 kL. In some embodiments, the biomanufacturing process comprises a production phase performed in a bioreactor with a capacity of about 10 KL. In some embodiments, the biomanufacturing process comprises a production phase performed in a bioreactor with a capacity of about 20 kL.

Also disclosed herein is a method for harvesting a recombinant protein during a biomanufacturing process comprising:
establishing a cell culture by inoculating a bioreactor with mammalian cells expressing the recombinant protein;
maintaining the cell culture during a growth phase and a production phase, wherein an exogenous protease inhibitor is admixed with the cell culture during the growth phase and/or during or after the production phase;
admixing an acidic solution with the cell culture to obtain an acidified cell culture with a pH of about 4.6 to about 5.3; and
incubating the acidified cell culture to induce agglomeration of one or more cell culture impurities,
wherein the recombinant protein comprises an asparaginyl endopeptidase (AEP) cleavage site.

In some embodiments, the exogenous protease inhibitor is an AEP inhibitor.

In some embodiments, an exogenous protease inhibitor is admixed with the cell culture before admixing with the acidic solution. In some embodiments, an exogenous protease inhibitor is admixed with the acidified cell culture. In some embodiments, the exogenous protease inhibitor is an AEP inhibitor.

In some embodiments, an exogenous protease inhibitor is added to a pool downstream of the acidified cell culture (such as, e.g., a protein A pool or a low pH virus inactivation pool). In some embodiments, the exogenous protease inhibitor is an AEP inhibitor.

In some embodiments, the method comprises incubating the acidified cell culture at a temperature of about 4° C. to about 37° C. In some embodiments, the method comprises incubating the acidified cell culture at a temperature of about 15° C. to about 20° C.

In some embodiments, the acidic solution comprises an acid selected from acetic acid, trichloroacetic acid, formic acid, phosphoric acid, sulfuric acid, citric acid, caprylic acid, and combinations of any of the foregoing. In some embodiments, the acidic solution comprises an acid selected from acetic acid, phosphoric acid, and combinations thereof. In some embodiments, the acidic solution comprises sulfuric acid. In some embodiments, the acidic solution comprises citric acid. In some embodiments, the acidic solution comprises acetic acid. In some embodiments, the acidic solution comprises 1 M acetic acid. In some embodiments, the acidic solution comprises phosphoric acid. In some embodiments, the acidic solution comprises 2 M phosphoric acid.

In some embodiments, the method comprises incubating the acidified cell culture for at least about 30 minutes. In some embodiments, the method comprises incubating the acidified cell culture for at least about 40 minutes. In some embodiments, the method comprises incubating the acidified cell culture for at least about 45 minutes. In some embodiments, the method comprises incubating the acidified cell culture for at least about 60 minutes.

In some embodiments, the method comprises incubating the acidified cell culture for about 5 minutes to about 48 hours. In some embodiments, the method comprises incubating the acidified cell culture for about 5 minutes to about 24 hours. In some embodiments, the method comprises incubating the acidified cell culture for about 5 minutes to about 12 hours. In some embodiments, the method comprises incubating the acidified cell culture for about 5 minutes to about 120 minutes. In some embodiments, the method comprises incubating the acidified cell culture for about 5 minutes to about 90 minutes. In some embodiments, the method comprises incubating the acidified cell culture for about 5 minutes to about 60 minutes. In some embodiments, the method comprises incubating the acidified cell culture for about 30 minutes to about 90 minutes.

In some embodiments, the method further comprises separating the one or more cell culture impurities from the acidified cell culture to obtain a clarified cell culture. In some embodiments, the one or more cell culture impurities are separated from the acidified cell culture by centrifugation and/or depth filtration. In some embodiments, the one or more cell culture impurities are separated from the acidified cell culture by centrifugation. In some embodiments, the one or more cell culture impurities are separated from the acidified cell culture by continuous solid discharge centrifugation, disk stack centrifugation, and/or depth filtration. In some embodiments, the one or more cell culture impurities are separated from the acidified cell culture by continuous solid discharge centrifugation. In some embodiments, the one or more cell culture impurities are separated from the acidified cell culture by disk stack centrifugation. In some embodiments, the one or more cell culture impurities are separated from the acidified cell culture by intermittent discharge centrifugation. In some embodiments, the one or more cell culture impurities are separated from the acidified cell culture by depth filtration. In some embodiments, the one or more cell culture impurities are separated from the acidified cell culture by depth filtration without centrifugation. In some embodiments, the one or more cell culture impurities are separated from the acidified cell culture by continuous solid discharge centrifugation and depth filtration.

In some embodiments, the depth filtration is performed at a loading of less than about 150 L/m². In some embodiments, the depth filtration is performed at a loading of less than about 140 L/m². In some embodiments, the depth filtration is performed at a loading of less than about 130 L/m². In some embodiments, the depth filtration is performed at a loading of less than about 120 L/m². In some embodiments, the depth filtration is performed at a loading of less than about 110 L/m². In some embodiments, the depth filtration is performed at a loading of less than about 100 L/m². In some embodiments, the depth filtration is performed at a loading of less than about 95 L/m². In some embodiments, the depth filtration is performed at a loading of less than about 90 L/m². In some embodiments, the depth filtration is performed at a loading of less than about 85 L/m². In some embodiments, the depth filtration is performed at a loading of less than about 80 L/m². In some embodiments, the depth filtration is performed at a loading of less than about 75 L/m².

In some embodiments, the method comprises neutralizing the acidified cell culture prior to separating the one or more cell culture impurities from the acidified cell culture. In some embodiments, the method does not comprise neutralizing the acidified cell culture prior to separating the one or more cell culture impurities from the acidified cell culture.

In some embodiments, the method comprises neutralizing the clarified cell culture.

In some embodiments, the one or more cell culture impurities comprise cell culture debris, host cell proteins, and nucleic acids. In some embodiments, the one or more cell culture impurities comprise cell culture debris. In some embodiments, the one or more cell culture impurities comprise host cell proteins. In some embodiments, the one or more cell culture impurities comprise nucleic acids. In some embodiments, the one or more cell culture impurities are selected from host cell proteins and nucleic acids.

In some embodiments, the pH of the acidified cell culture is about 4.7 to about 5.3. In some embodiments, the pH of the acidified cell culture is about 4.8 to about 5.3. In some embodiments, the pH of the acidified cell culture is about 4.9 to about 5.3. In some embodiments, the pH of the acidified cell culture is about 5.0 to about 5.3. In some embodiments, the pH of the acidified cell culture is about 4.7 to about 5.2. In some embodiments, the pH of the acidified cell culture is about 4.8 to about 5.2. In some embodiments, the pH of the acidified cell culture is about 4.9 to about 5.2. In some embodiments, the pH of the acidified cell culture is about 5.0 to about 5.2.

In some embodiments, the pH of the acidified cell culture is about 4.6. In some embodiments, the pH of the acidified cell culture is about 4.7. In some embodiments, the pH of the acidified cell culture is about 4.8. In some embodiments, the pH of the acidified cell culture is about 4.9. In some embodiments, the pH of the acidified cell culture is about 5.0. In some embodiments, the pH of the acidified cell culture is about 5.1. In some embodiments, the pH of the acidified cell culture is about 5.2. In some embodiments, the pH of the acidified cell culture is about 5.3.

In some embodiments, the pH of the acidified cell culture deviates by less than about ±0.2 pH units during the incubation. In some embodiments, the pH of the acidified cell culture deviates by less than about ±0.1 pH units during the incubation.

In some embodiments, the pH of the acidified cell culture deviates by less than about 25% during the incubation. In some embodiments, the pH of the acidified cell culture deviates by less than about 20% during the incubation. In some embodiments, the pH of the acidified cell culture deviates by less than about 15% during the incubation. In some embodiments, the pH of the acidified cell culture deviates by less than about 10% during the incubation. In some embodiments, the pH of the acidified cell culture deviates by less than about 5% during the incubation.

In some embodiments, the acidified cell culture comprises recombinant protein comprising a sequence upstream of the AEP cleavage site and a sequence downstream of the AEP cleavage site.

In some embodiments, the acidified cell culture comprises a reduced quantity of one or more fragments of the recombinant protein relative to an alternative acidified cell culture with a pH of less than about 4.6. In some embodiments, the fragments of the recombinant protein comprise species cleaved at the AEP cleavage site.

In some embodiments, less than about 5% w/w of the recombinant protein in the acidified cell culture is fragments of the recombinant protein. In some embodiments, less than about 4% w/w of the recombinant protein in the acidified cell culture is fragments of the recombinant protein. In some embodiments, less than about 3% w/w of the recombinant protein in the acidified cell culture is fragments of the recombinant protein. In some embodiments, less than about 2% w/w of the recombinant protein in the acidified cell culture is fragments of the recombinant protein. In some embodiments, less than about 1% w/w of the recombinant protein in the acidified cell culture is fragments of the recombinant protein. Fragment content may be assessed, e.g., via UV absorbance relative peak area on reduced capillary electrophoresis.

In some embodiments, the method further comprises measuring AEP quantity or activity prior to the admixing. In some embodiments, the method further comprises measuring AEP quantity prior to the admixing. In some embodiments, the method further comprises measuring AEP activity prior to the admixing. In some embodiments, the method further comprises measuring AEP proenzyme quantity prior to the admixing.

In some embodiments, the AEP or AEP proenzyme quantity is measured using mass spectrometry or an immunoassay. In some embodiments, the AEP or AEP proenzyme quantity is measured using mass spectrometry. In some embodiments, the AEP or AEP proenzyme quantity is measured using an immunoassay.

In some embodiments, the mammalian cells are CHO cells and the AEP proenzyme quantity is measured by subjecting a pool sample to trypsin to obtain a trypsin-digest sample and measuring the amount of LMSTNDLK (SEQ ID NO: 1) and/or LDLTPSPEVPLTILK (SEQ ID NO: 2) in the trypsin-digest sample. In some embodiments, the method further comprises comparing the measured amount of SEQ ID NO: 1 and/or SEQ ID NO: 2 to one or more benchmark values, such as, e.g., a measured amount in a trypsin-digest sample comprising a known amount of AEP at a pH greater than or equal to 5.5.

In some embodiments, the AEP activity is measured using a cleavage assay.

In some embodiments, the mammalian cells are CHO cells.

In some embodiments, the recombinant protein is an antigen-binding protein. In some embodiments, the recombinant protein is an antibody. In some embodiments, the recombinant protein is an IgG2 antibody. In some embodiments, the recombinant protein comprises an IgG2 heavy chain constant region. In some embodiments, the recombinant protein comprises a wild-type IgG2 heavy chain constant region. In some embodiments, the recombinant protein comprises an engineered IgG2 heavy chain constant region.

In some embodiments, the recombinant protein comprises a N192/F193 sequence motif in a CH1 domain of a heavy chain constant region, according to EU numbering.

In some embodiments, the biomanufacturing process comprises a perfusion cell culture process, a concentrated fed batch cell culture process, or an intensified cell culture process. In some embodiments, the biomanufacturing process comprises a perfusion cell culture process. In some embodiments, the biomanufacturing process comprises a concentrated fed batch cell culture process. In some embodiments, the biomanufacturing process comprises an intensified cell culture process.

In some embodiments, the biomanufacturing process comprises a production phase with a viable cell density of at least about 10⁶ cells/mL. In some embodiments, the biomanufacturing process comprises a production phase with a viable cell density of at least about 5×10⁶ cells/mL. In some embodiments, the biomanufacturing process comprises a production phase with a viable cell density of at least about 10⁷ cells/mL.

In some embodiments, the biomanufacturing process comprises a production phase with a packed cell volume of about 2% to about 40%. In some embodiments, the biomanufacturing process comprises a production phase with a packed cell volume of about 15% to about 18%. In some embodiments, the biomanufacturing process comprises a production phase with a packed cell volume of about 25% to about 35%. In some embodiments, the biomanufacturing process comprises a production phase with a packed cell volume of about 3% to about 15%.

In some embodiments, the biomanufacturing process comprises a production phase performed in a bioreactor with a capacity of at least about 500 L. In some embodiments, the biomanufacturing process comprises a production phase performed in a bioreactor with a capacity of at least about 1 kL. In some embodiments, the biomanufacturing process comprises a production phase performed in a bioreactor with a capacity of at least about 2 kL. In some embodiments, the biomanufacturing process comprises a production phase performed in a bioreactor with a capacity of at least about 5 kL. In some embodiments, the biomanufacturing process comprises a production phase performed in a bioreactor with a capacity of at least about 10 KL. In some embodiments, the biomanufacturing process comprises a production phase performed in a bioreactor with a capacity of at least about 20 kL.

In some embodiments, the biomanufacturing process comprises a production phase performed in a bioreactor with a capacity of about 500 L. In some embodiments, the biomanufacturing process comprises a production phase performed in a bioreactor with a capacity of about 1 kL. In some embodiments, the biomanufacturing process comprises a production phase performed in a bioreactor with a capacity of about 2 kL. In some embodiments, the biomanufacturing process comprises a production phase performed in a bioreactor with a capacity of about 5 kL. In some embodiments, the biomanufacturing process comprises a production phase performed in a bioreactor with a capacity of about 10 KL. In some embodiments, the biomanufacturing process comprises a production phase performed in a bioreactor with a capacity of about 20 kL.

Also disclosed herein is a method for inhibiting cleavage of a recombinant protein comprising an asparaginyl endopeptidase (AEP) cleavage site during a biomanufacturing process comprising an acid precipitation, the method comprising:
measuring a quantity or activity of AEP in a sample isolated from a cell culture, harvest, or purification operation of the biomanufacturing process; and
adjusting the acid precipitation pH to about 4.6 to about 5.3 if the quantity or activity of AEP is above a threshold.

In some embodiments, the method comprises adjusting the acid precipitation pH to about 4.7 to about 5.3. In some embodiments, the method comprises adjusting the acid precipitation pH to about 4.8 to about 5.3. In some embodiments, the method comprises adjusting the acid precipitation pH to about 4.9 to about 5.3. In some embodiments, the method comprises adjusting the acid precipitation pH to about 5.0 to about 5.3. In some embodiments, the method comprises adjusting the acid precipitation pH to about 4.7 to about 5.2. In some embodiments, the method comprises adjusting the acid precipitation pH to about 4.8 to about 5.2. In some embodiments, the method comprises adjusting the acid precipitation pH to about 4.9 to about 5.2. In some embodiments, the method comprises adjusting the acid precipitation pH to about 5.0 to about 5.2.

In some embodiments, the method comprises adjusting the acid precipitation pH to about 4.6. In some embodiments, the method comprises adjusting the acid precipitation pH to about 4.7. In some embodiments, the method comprises adjusting the acid precipitation pH to about 4.8. In some embodiments, the method comprises adjusting the acid precipitation pH to about 4.9. In some embodiments, the method comprises adjusting the acid precipitation pH to about 5.0. In some embodiments, the method comprises adjusting the acid precipitation pH to about 5.1. In some embodiments, the method comprises adjusting the acid precipitation pH to about 5.2. In some embodiments, the method comprises adjusting the acid precipitation pH to about 5.3.

In some embodiments, adjusting the acid precipitation pH comprises admixing a cell culture with an acidic solution. In some embodiments, the acidic solution comprises an acid selected from acetic acid, trichloroacetic acid, formic acid, phosphoric acid, sulfuric acid, citric acid, caprylic acid, and combinations of any of the foregoing. In some embodiments, the acidic solution comprises an acid selected from acetic acid, phosphoric acid, and combinations thereof. In some embodiments, the acidic solution comprises sulfuric acid. In some embodiments, the acidic solution comprises citric acid. In some embodiments, the acidic solution comprises acetic acid. In some embodiments, the acidic solution comprises 1 M acetic acid. In some embodiments, the acidic solution comprises phosphoric acid. In some embodiments, the acidic solution comprises 2 M phosphoric acid.

In some embodiments, the acid precipitation is performed for least about 30 minutes. In some embodiments, the acid precipitation is performed for at least about 40 minutes. In some embodiments, the acid precipitation is performed for at least about 45 minutes. In some embodiments, the acid precipitation is performed for at least about 60 minutes.

In some embodiments, the acid precipitation is performed for about 5 minutes to about 24 hours. In some embodiments, the acid precipitation is performed for about 5 minutes to about 12 hours. In some embodiments, the acid precipitation is performed for about 5 minutes to about 120 minutes. In some embodiments, the acid precipitation is performed for about 5 minutes to about 90 minutes. In some embodiments, the acid precipitation is performed for about 5 minutes to about 60 minutes. In some embodiments, the acid precipitation is performed for about 30 minutes to about 90 minutes.

In some embodiments, cleavage of the recombinant protein is inhibited relative to a recombinant protein produced by an alternative biomanufacturing process using an acid precipitation pH of less than about 4.6. In some embodiments, cleavage of the recombinant protein is inhibited by at least about 25% relative to a recombinant protein produced by an alternative biomanufacturing process using an acid precipitation pH of less than about 4.6. In some embodiments, cleavage of the recombinant protein is inhibited by at least about 50% relative to a recombinant protein produced by an alternative biomanufacturing process using an acid precipitation pH of less than about 4.6. In some embodiments, cleavage of the recombinant protein is inhibited by at least about 75% relative to a recombinant protein produced by an alternative biomanufacturing process using an acid precipitation pH of less than about 4.6.

In some embodiments, the method comprises measuring a quantity of AEP. In some embodiments, the AEP quantity is measured using mass spectrometry or an immunoassay. In some embodiments, the AEP quantity is measured using mass spectrometry. In some embodiments, the AEP quantity is measured using an immunoassay.

In some embodiments, the threshold for AEP quantity is greater than about 500 ng AEP per 1 mg of harvest cell culture fluid (HCCF). In some embodiments, the threshold for AEP quantity is greater than about 750 ng AEP per 1 mg of HCCF. In some embodiments, the threshold for AEP quantity is greater than about 1000 ng AEP per 1 mg of HCCF. In some embodiments, the threshold for AEP quantity is greater than about 1500 ng AEP per 1 mg of HCCF. In some embodiments, the threshold for AEP quantity is greater than about 2000 ng AEP per 1 mg of HCCF.

In some embodiments, the threshold for AEP quantity is about 500 ng AEP per 1 mg of HCCF. In some embodiments, the threshold for AEP quantity is about 750 ng AEP per 1 mg of HCCF. In some embodiments, the threshold for AEP quantity is about 1000 ng AEP per 1 mg of HCCF. In some embodiments, the threshold for AEP quantity is about 1500 ng AEP per 1 mg of HCCF. In some embodiments, the threshold for AEP quantity is about 2000 ng AEP per 1 mg of HCCF.

In some embodiments, the method comprises measuring an activity of AEP. In some embodiments, the AEP activity is measured using a cleavage assay.

In some embodiments, the threshold for AEP activity is greater than about 1% clipping of the recombinant protein at the AEP cleavage site. In some embodiments, the threshold for AEP activity is greater than about 2% clipping of the recombinant protein at the AEP cleavage site. In some embodiments, the threshold for AEP activity is greater than about 3% clipping of the recombinant protein at the AEP cleavage site. In some embodiments, the threshold for AEP activity is greater than about 4% clipping of the recombinant protein at the AEP cleavage site. In some embodiments, the threshold for AEP activity is greater than about 5% clipping of the recombinant protein at the AEP cleavage site.

In some embodiments, the sample is isolated from a cell culture or harvest operation. In some embodiments, the sample is isolated from a cell culture operation. In some embodiments, the sample is isolated from a harvest operation.

In some embodiments, the method further comprises adjusting a dissolved oxygen level in the cell culture to about 64 mmHg to about 128 mmHg during a cell culture cooling operation of the biomanufacturing process if the quantity or activity of AEP is above the threshold.

In some embodiments, the method further comprises admixing an exogenous protease inhibitor with a cell culture during a growth phase and/or a production phase of the biomanufacturing process if the quantity or activity of AEP is above the threshold. In some embodiments, the exogenous protease inhibitor is an AEP inhibitor.

In some embodiments, an exogenous protease inhibitor is added to a pool downstream of the acidified cell culture (such as, e.g., a protein A pool or a low pH virus inactivation pool). In some embodiments, the exogenous protease inhibitor is an AEP inhibitor.

In some embodiments, the recombinant protein is an antigen-binding protein. In some embodiments, the recombinant protein is an antibody. In some embodiments, the recombinant protein is an IgG2 antibody. In some embodiments, the recombinant protein comprises an IgG2 heavy chain constant region. In some embodiments, the recombinant protein comprises a wild-type IgG2 heavy chain constant region. In some embodiments, the recombinant protein comprises an engineered IgG2 heavy chain constant region.

In some embodiments, the recombinant protein comprises a N192/F193 sequence motif in a CH1 domain of a heavy chain constant region, according to EU numbering.

In some embodiments, the biomanufacturing process comprises a cell culture process selected from a perfusion cell culture process, a concentrated fed batch cell culture process, and an intensified cell culture process, wherein the cell culture process utilizes mammalian cells. In some embodiments, the mammalian cells are selected from CHO cells. In some embodiments, the biomanufacturing process comprises a perfusion cell culture process. In some embodiments, the biomanufacturing process comprises a concentrated fed batch cell culture process. In some embodiments, the biomanufacturing process comprises an intensified cell culture process.

In some embodiments, the biomanufacturing process comprises a production phase with a viable cell density of at least about 10⁶ cells/mL. In some embodiments, the biomanufacturing process comprises a production phase with a viable cell density of at least about 5×10⁶ cells/mL. In some embodiments, the biomanufacturing process comprises a production phase with a viable cell density of at least about 10⁷ cells/mL.

In some embodiments, the biomanufacturing process comprises a production phase with a packed cell volume of about 2% to about 40%. In some embodiments, the biomanufacturing process comprises a production phase with a packed cell volume of about 15% to about 18%. In some embodiments, the biomanufacturing process comprises a production phase with a packed cell volume of about 25% to about 35%. In some embodiments, the biomanufacturing process comprises a production phase with a packed cell volume of about 3% to about 15%.

In some embodiments, the biomanufacturing process comprises a production phase performed in a bioreactor with a capacity of at least about 500 L. In some embodiments, the biomanufacturing process comprises a production phase performed in a bioreactor with a capacity of at least about 1 kL. In some embodiments, the biomanufacturing process comprises a production phase performed in a bioreactor with a capacity of at least about 2 kL. In some embodiments, the biomanufacturing process comprises a production phase performed in a bioreactor with a capacity of at least about 5 kL. In some embodiments, the biomanufacturing process comprises a production phase performed in a bioreactor with a capacity of at least about 10 KL. In some embodiments, the biomanufacturing process comprises a production phase performed in a bioreactor with a capacity of at least about 20 kL.

In some embodiments, the biomanufacturing process comprises a production phase performed in a bioreactor with a capacity of about 500 L. In some embodiments, the biomanufacturing process comprises a production phase performed in a bioreactor with a capacity of about 1 kL. In some embodiments, the biomanufacturing process comprises a production phase performed in a bioreactor with a capacity of about 2 kL. In some embodiments, the biomanufacturing process comprises a production phase performed in a bioreactor with a capacity of about 5 kL. In some embodiments, the biomanufacturing process comprises a production phase performed in a bioreactor with a capacity of about 10 kL. In some embodiments, the biomanufacturing process comprises a production phase performed in a bioreactor with a capacity of about 20 kL.

Also disclosed herein is a method for inhibiting cleavage of a recombinant protein comprising an asparaginyl endopeptidase (AEP) cleavage site during a biomanufacturing process comprising an acid precipitation, the method comprising:
measuring a quantity of one or more fragments of the recombinant protein in a sample isolated during a cell culture, harvest, or purification operation of the biomanufacturing process; and
adjusting the acid precipitation pH to about 4.6 to about 5.3 if the quantity of the one or more fragments is above a threshold.

In some embodiments, the threshold is at least about 1% w/w of the recombinant protein is the one or more fragments. In some embodiments, the threshold is at least about 2% w/w of the recombinant protein is the one or more fragments. In some embodiments, the threshold is at least about 3% w/w of the recombinant protein is the one or more fragments. In some embodiments, the threshold is at least about 4% w/w of the recombinant protein is the one or more fragments. In some embodiments, the threshold is at least about 5% w/w of the recombinant protein is the one or more fragments. Fragment content may be assessed, e.g., via UV absorbance relative peak area on reduced capillary electrophoresis.

In some embodiments, the one or more fragments comprise species cleaved at the AEP cleavage site.

In some embodiments, the method comprises adjusting the acid precipitation pH to about 4.7 to about 5.3. In some embodiments, the method comprises adjusting the acid precipitation pH to about 4.8 to about 5.3. In some embodiments, the method comprises adjusting the acid precipitation pH to about 4.9 to about 5.3. In some embodiments, the method comprises adjusting the acid precipitation pH to about 5.0 to about 5.3. In some embodiments, the method comprises adjusting the acid precipitation pH to about 4.7 to about 5.2. In some embodiments, the method comprises adjusting the acid precipitation pH to about 4.8 to about 5.2. In some embodiments, the method comprises adjusting the acid precipitation pH to about 4.9 to about 5.2. In some embodiments, the method comprises adjusting the acid precipitation pH to about 5.0 to about 5.2.

In some embodiments, the method comprises adjusting the acid precipitation pH to about 4.6. In some embodiments, the method comprises adjusting the acid precipitation pH to about 4.7. In some embodiments, the method comprises adjusting the acid precipitation pH to about 4.8. In some embodiments, the method comprises adjusting the acid precipitation pH to about 4.9. In some embodiments, the method comprises adjusting the acid precipitation pH to about 5.0. In some embodiments, the method comprises adjusting the acid precipitation pH to about 5.1. In some embodiments, the method comprises adjusting the acid precipitation pH to about 5.2. In some embodiments, the method comprises adjusting the acid precipitation pH to about 5.3.

In some embodiments, adjusting the acid precipitation pH comprises admixing a cell culture with an acidic solution. In some embodiments, the acidic solution comprises an acid selected from acetic acid, trichloroacetic acid, formic acid, phosphoric acid, sulfuric acid, citric acid, caprylic acid, and combinations of any of the foregoing. In some embodiments, the acidic solution comprises an acid selected from acetic acid, phosphoric acid, and combinations thereof. In some embodiments, the acidic solution comprises sulfuric acid. In some embodiments, the acidic solution comprises citric acid. In some embodiments, the acidic solution comprises acetic acid. In some embodiments, the acidic solution comprises 1 M acetic acid. In some embodiments, the acidic solution comprises phosphoric acid. In some embodiments, the acidic solution comprises 2 M phosphoric acid.

In some embodiments, the acid precipitation is performed for least about 30 minutes. In some embodiments, the acid precipitation is performed for at least about 40 minutes. In some embodiments, the acid precipitation is performed for at least about 45 minutes. In some embodiments, the acid precipitation is performed for at least about 60 minutes.

In some embodiments, the acid precipitation is performed for about 5 minutes to about 24 hours. In some embodiments, the acid precipitation is performed for about 5 minutes to about 12 hours. In some embodiments, the acid precipitation is performed for about 5 minutes to about 120 minutes. In some embodiments, the acid precipitation is performed for about 5 minutes to about 90 minutes. In some embodiments, the acid precipitation is performed for about 5 minutes to about 60 minutes. In some embodiments, the acid precipitation is performed for about 30 minutes to about 90 minutes.

In some embodiments, cleavage of the recombinant protein is inhibited relative to a recombinant protein produced by an alternative biomanufacturing process using an acid precipitation pH of less than about 4.6. In some embodiments, cleavage of the recombinant protein is inhibited by at least about 25% relative to a recombinant protein produced by an alternative biomanufacturing process using an acid precipitation pH of less than about 4.6. In some embodiments, cleavage of the recombinant protein is inhibited by at least about 50% relative to a recombinant protein produced by an alternative biomanufacturing process using an acid precipitation pH of less than about 4.6. In some embodiments, cleavage of the recombinant protein is inhibited by at least about 75% relative to a recombinant protein produced by an alternative biomanufacturing process using an acid precipitation pH of less than about 4.6.

In some embodiments, the sample is isolated from a cell culture or harvest operation. In some embodiments, the sample is isolated from a cell culture operation. In some embodiments, the sample is isolated from a harvest operation.

In some embodiments, the method further comprises adjusting a dissolved oxygen level in a cell culture to about 64 mmHg to about 128 mmHg during a cell culture cooling operation of the biomanufacturing process if the quantity of the one or more fragments is above the threshold.

In some embodiments, the method further comprises admixing an exogenous protease inhibitor with a cell culture during a growth phase and/or a production phase of the biomanufacturing process if the quantity of the one or more fragments is above the threshold. In some embodiments, the exogenous protease inhibitor is an AEP inhibitor.

In some embodiments, the recombinant protein is an antigen-binding protein. In some embodiments, the recombinant protein is an antibody. In some embodiments, the recombinant protein is an IgG2 antibody. In some embodiments, the recombinant protein comprises an IgG2 heavy chain constant region. In some embodiments, the recombinant protein comprises a wild-type IgG2 heavy chain constant region. In some embodiments, the recombinant protein comprises an engineered IgG2 heavy chain constant region.

In some embodiments, the recombinant protein comprises a N192/F193 sequence motif in a CH1 domain of a heavy chain constant region, according to EU numbering.

In some embodiments, the biomanufacturing process comprises a cell culture process selected from a perfusion cell culture process, a concentrated fed batch cell culture process, and an intensified cell culture process, wherein the cell culture process utilizes mammalian cells. In some embodiments, the mammalian cells are selected from CHO cells. In some embodiments, the biomanufacturing process comprises a perfusion cell culture process. In some embodiments, the biomanufacturing process comprises a concentrated fed batch cell culture process. In some embodiments, the biomanufacturing process comprises an intensified cell culture process.

In some embodiments, the biomanufacturing process comprises a production phase with a viable cell density of at least about 10⁶ cells/mL. In some embodiments, the biomanufacturing process comprises a production phase with a viable cell density of at least about 5×10⁶ cells/mL. In some embodiments, the biomanufacturing process comprises a production phase with a viable cell density of at least about 10⁷ cells/mL.

In some embodiments, the biomanufacturing process comprises a production phase with a packed cell volume of about 2% to about 40%. In some embodiments, the biomanufacturing process comprises a production phase with a packed cell volume of about 15% to about 18%. In some embodiments, the biomanufacturing process comprises a production phase with a packed cell volume of about 25% to about 35%. In some embodiments, the biomanufacturing process comprises a production phase with a packed cell volume of about 3% to about 15%.

In some embodiments, the biomanufacturing process comprises a production phase performed in a bioreactor with a capacity of at least about 500 L. In some embodiments, the biomanufacturing process comprises a production phase performed in a bioreactor with a capacity of at least about 1 kL. In some embodiments, the biomanufacturing process comprises a production phase performed in a bioreactor with a capacity of at least about 2 kL. In some embodiments, the biomanufacturing process comprises a production phase performed in a bioreactor with a capacity of at least about 5 kL. In some embodiments, the biomanufacturing process comprises a production phase performed in a bioreactor with a capacity of at least about 10 kL. In some embodiments, the biomanufacturing process comprises a production phase performed in a bioreactor with a capacity of at least about 20 kL.

In some embodiments, the biomanufacturing process comprises a production phase performed in a bioreactor with a capacity of about 500 L. In some embodiments, the biomanufacturing process comprises a production phase performed in a bioreactor with a capacity of about 1 kL. In some embodiments, the biomanufacturing process comprises a production phase performed in a bioreactor with a capacity of about 2 kL. In some embodiments, the biomanufacturing process comprises a production phase performed in a bioreactor with a capacity of about 5 kL. In some embodiments, the biomanufacturing process comprises a production phase performed in a bioreactor with a capacity of about 10 kL. In some embodiments, the biomanufacturing process comprises a production phase performed in a bioreactor with a capacity of about 20 kL.

Also disclosed herein is a method for monitoring activation of AEP in a sample from a biomanufacturing process utilizing CHO cells, wherein the method comprises subjecting the sample to trypsin to obtain a trypsin-digest sample and measuring the amount of LMSTNDLK (SEQ ID NO: 1) and/or LDLTPSPEVPLTILK (SEQ ID NO: 2) in the trypsin-digest sample. In some embodiments, the method further comprises comparing the measured amount of SEQ ID NO: 1 and/or SEQ ID NO: 2 to one or more benchmark values, such as, e.g., a measured amount in a trypsin-digest sample comprising a known amount of AEP at a pH greater than or equal to 5.5.

### Non-Limiting Example Features

Without limitation, some example embodiments/features of the present disclosure include:
E1. A method for harvesting a recombinant protein during a biomanufacturing process comprising:
   admixing an acidic solution with a cell culture comprising the recombinant protein to obtain an acidified cell culture with a pH of about 4.6 to about 5.3; and
   incubating the acidified cell culture to induce agglomeration of one or more cell culture impurities,
   wherein the recombinant protein comprises an asparaginyl endopeptidase (AEP) cleavage site.
E2. A method for harvesting a recombinant protein during a biomanufacturing process comprising:
   establishing a cell culture by inoculating a bioreactor with mammalian cells expressing the recombinant protein;
   maintaining the cell culture during a growth phase and a production phase;
   cooling the cell culture, wherein a dissolved oxygen level of about 64 mmHg to about 128 mmHg is maintained in the cell culture during the cooling;
   admixing an acidic solution with the cell culture to obtain an acidified cell culture with a pH of about 4.6 to about 5.3; and
   incubating the acidified cell culture to induce agglomeration of one or more cell culture impurities,
   wherein the recombinant protein comprises an asparaginyl endopeptidase (AEP) cleavage site.
E3. The method according to E2, wherein the cell culture is cooled to about 4° C. to about 15° C. (e.g., about 9° C. to about 11° C.).
E4. The method according to E2 or E3, wherein the cell culture is cooled to about 10° C.
E5. The method according to any one of E2 to E4, wherein an exogenous protease inhibitor is admixed with the cell culture during the growth phase, the production phase, and/or the cooling phase.
E6. A method for harvesting a recombinant protein during a biomanufacturing process comprising:
   establishing a cell culture by inoculating a bioreactor with mammalian cells expressing the recombinant protein;
   maintaining the cell culture during a growth phase and a production phase, wherein an exogenous protease inhibitor is admixed with the cell culture during the growth phase and/or during or after the production phase;
   admixing an acidic solution with the cell culture to obtain an acidified cell culture with a pH of about 4.6 to about 5.3; and
   incubating the acidified cell culture to induce agglomeration of one or more cell culture impurities,
   wherein the recombinant protein comprises an asparaginyl endopeptidase (AEP) cleavage site.
E7. The method according to E5 or E6, wherein the exogenous protease inhibitor is an AEP inhibitor.
E8. The method according to any one of E1 to E7, wherein the acidic solution comprises an acid selected from acetic acid, trichloroacetic acid, formic acid, phosphoric acid, sulfuric acid, citric acid, caprylic acid, and combinations of any of the foregoing.
E9. The method according to any one of E1 to E8, comprising incubating the acidified cell culture for at least about 60 minutes.
E10. The method according to any one of E1 to E9, comprising incubating the acidified cell culture for about 60 minutes to about 48 hours.
E11. The method according to any one of E1 to E10, further comprising separating the one or more cell culture impurities from the acidified cell culture to obtain a clarified cell culture.
E12. The method according to E11, wherein the one or more cell culture impurities are separated from the acidified cell culture by continuous solid discharge centrifugation, disk stack centrifugation, and/or depth filtration.
E13. The method according to E12, wherein the depth filtration is performed at a loading of less than about 150 L/m² (e.g., less than about 125 L/m²).
E14. The method according to any one of E1 to E13, wherein the one or more cell culture impurities are selected from host cell proteins and nucleic acids.
E15. The method according to any one of E1 to E14, wherein the pH of the acidified cell culture is about 4.9 to about 5.2.
E16. The method according to any one of E1 to E15, wherein the pH of the acidified cell culture is about 5.0 to about 5.2.
E17. The method according to any one of E1 to E16, wherein the pH of the acidified cell culture is about 5.1.
E18. The method according to any one of E1 to E17, wherein the pH of the acidified cell culture deviates by less than about ±0.2 pH units during the incubation.
E19. The method according to any one of E1 to E18, wherein the acidified cell culture comprises recombinant protein comprising a sequence upstream of the AEP cleavage site and a sequence downstream of the AEP cleavage site.
E20. The method according to any one of E1 to E19, wherein the acidified cell culture comprises a reduced quantity of one or more fragments of the recombinant protein relative to an alternative acidified cell culture with a pH of less than about 4.6.
E21. The method according to any one of E1 to E20, wherein less than about 5% w/w (e.g., less than about 4% w/w; less than about 3% w/w; less than about 2% w/w) of the recombinant protein in the acidified cell culture is fragments of the recombinant protein, e.g., as detected by UV absorbance relative peak area on reduced capillary electrophoresis.
E22. The method according to any one of E1 to E19, wherein less than about 1% w/w of the recombinant protein in the acidified cell culture is fragments of the recombinant protein.
E23. The method according to E21 or E22, wherein the fragments of the recombinant protein comprise species cleaved at the AEP cleavage site.
E24. The method according to any one of E1 to E23, further comprising measuring AEP quantity or activity prior to the admixing.
E25. The method according to any one of E1 to E23, further comprising measuring AEP proenzyme quantity prior to the admixing.
E26. The method according to E24 or E25, wherein the AEP or AEP proenzyme quantity is measured using mass spectrometry or an immunoassay.
E27. The method according to E24, wherein the AEP activity is measured using a cleavage assay.
E28. A method for inhibiting cleavage of a recombinant protein comprising an asparaginyl endopeptidase (AEP) cleavage site during a biomanufacturing process comprising an acid precipitation, the method comprising:
   measuring a quantity or activity of AEP in a sample isolated from a cell culture, harvest, or purification operation of the biomanufacturing process; and
   adjusting the acid precipitation pH to about 4.6 to about 5.3 if the quantity or activity of AEP is above a threshold.
E29. The method according to E28, wherein cleavage of the recombinant protein is inhibited relative to a recombinant protein produced by an alternative biomanufacturing process using an acid precipitation pH of less than about 4.6.
E30. The method according to E28 or E29, wherein the sample is isolated from a cell culture or harvest operation.
E31. The method according to any one of E28 to E30, wherein the AEP quantity is measured using mass spectrometry or an immunoassay.
E32. The method according to any one of E28 to E30, wherein the AEP activity is measured using a cleavage assay.
E33. The method according to any one of E28 to E32, further comprising adjusting a dissolved oxygen level in the cell culture to about 64 mmHg to about 128 mmHg during a cell culture cooling operation of the biomanufacturing process if the quantity or activity of AEP is above the threshold.
E34. The method according to any one of E28 to E33, further comprising admixing an exogenous protease inhibitor (e.g., an exogenous AEP inhibitor) with a cell culture during a growth phase and/or a production phase of the biomanufacturing process if the quantity or activity of AEP is above the threshold.
E35. The method according to any one of E28 to E34, wherein the threshold for AEP quantity is greater than about 1000 ng AEP per 1 mg of harvest cell culture fluid (HCCF).
E36. The method according to any one of E28 to E35, wherein the threshold for AEP quantity is greater than about 2000 ng AEP per 1 mg of harvest cell culture fluid (HCCF).
E37. The method according to any one of E28 to E36, wherein the threshold for AEP activity is greater than about 3% clipping of the recombinant protein at the AEP cleavage site.
E38. A method for inhibiting cleavage of a recombinant protein comprising an asparaginyl endopeptidase (AEP) cleavage site during a biomanufacturing process comprising an acid precipitation, the method comprising:
   measuring a quantity of one or more fragments of the recombinant protein in a sample isolated during a cell culture, harvest, or purification operation of the biomanufacturing process; and
   adjusting the acid precipitation pH to about 4.6 to about 5.3 if the quantity of the one or more fragments is above a threshold.
E39. The method according to E38, wherein cleavage of the recombinant protein is inhibited relative to a recombinant protein produced by an alternative biomanufacturing process using an acid precipitation pH of less than about 4.6.
E40. The method according to E38 or E39, wherein the sample is isolated during a cell culture or harvest operation.
E41. The method according to any one of E38 to E40, further comprising adjusting a dissolved oxygen level in a cell culture to about 64 mmHg to about 128 mmHg during a cell culture cooling operation of the biomanufacturing process if the quantity of the one or more fragments is above the threshold.
E42. The method according to any one of E38 to E41, further comprising admixing an exogenous protease inhibitor (e.g., an exogenous AEP inhibitor) with a cell culture during a growth phase and/or a production phase of the biomanufacturing process if the quantity of the one or more fragments is above the threshold.
E43. The method according to any one of E38 to E42, wherein the threshold is at least about 1% w/w of the recombinant protein is the one or more fragments.
E44. The method according to any one of E38 to E43, wherein the acid precipitation pH is about 4.9 to about 5.2.
E45. The method according to any one of E38 to E44, wherein the acid precipitation pH is about 5.0 to about 5.2.
E46. The method according to any one of E38 to E45, wherein the acid precipitation pH is about 5.1.
E47. The method according to any one of E1 to E46, wherein the recombinant protein is an antigen-binding protein.
E48. The method according to any one of E1 to E47, wherein the recombinant protein is an antibody.
E49. The method according to any one of E1 to E48, wherein the recombinant protein is an IgG2 antibody.
E50. The method according to any one of E1 to E49, wherein the recombinant protein comprises an IgG2 heavy chain constant region.
E51. The method according to any one of E1 to E50, wherein the recombinant protein comprises a wild-type IgG2 heavy chain constant region.
E52. The method according to any one of E1 to E50, wherein the recombinant protein comprises an engineered IgG2 heavy chain constant region.
E53. The method according to any one of E1 to E52, wherein the recombinant protein comprises a CH1 domain of an IgG2 heavy chain constant region.
E54. The method according to any one of E1 to E53, wherein the recombinant protein comprises a N192/F193 sequence motif in a CH1 domain of a heavy chain constant region, according to EU numbering.
E55. The method according to any one of E1 to E54, wherein the biomanufacturing process comprises a perfusion cell culture process.
E56. The method according to any one of E1 to E54, wherein the biomanufacturing process comprises a concentrated fed batch cell culture process.
E57. The method according to any one of E1 to E54, wherein the biomanufacturing process comprises an intensified cell culture process.
E58. The method according to any one of E1 to E57, wherein the biomanufacturing process comprises a production phase with a viable cell density of at least about 10⁶ cells/mL.
E59. The method according to any one of E1 to E58, wherein the biomanufacturing process comprises a production phase with a viable cell density of at least about 10⁷ cells/mL.
E60. The method according to any one of E1 to E59, wherein the biomanufacturing process comprises a production phase with a packed cell volume of about 2% to about 40%.
E61. The method according to any one of E1 to E60, wherein the biomanufacturing process comprises a production phase performed in a bioreactor with a capacity of at least about 500 L.
E62. The method according to any one of E1 to E61, wherein the biomanufacturing process comprises a production phase performed in a bioreactor with a capacity of at least about 2 kL.

### BRIEF DESCRIPTION OF THE DRAWINGS

FIG. 1 shows AEP levels from in-process samples taken during a pilot-scale production of an IgG2 antibody (mAb1), where the process included an acid precipitation step at a pH of approximately 4.6.

FIG. 2 shows fragmentation levels as measured by LC-MS. The intact peptides (LMSTN³²⁵DLK (SEQ ID NO: 1) and LD³⁰⁵LTPSPE³¹¹VPLTILK (SEQ ID NO: 2)) reflect the proenzyme form of AEP. After acid precipitation, the intensities of these peptides decreased by about 100-fold, indicating that AEP was cleaved into its mature and active form at N³²⁵, D³⁰⁵ and E³¹¹.

FIG. 3 shows mAb1 fragmentation as measured by LC-MS.

### DETAILED DESCRIPTION

### Definitions

In some embodiments, “about,” when used in connection with a measurable numerical variable, refers to the indicated value of the variable and to all values of the variable that are within the experimental error of the indicated value (e.g., within the 95% confidence interval for the mean) or ±10% of the indicated value, whichever is greater. In some embodiments, numeric ranges are inclusive of the numbers defining the range (i.e., the endpoints).

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges may independently be included in the smaller ranges also encompassed within the disclosure, subject to any specifically excluded limit in the stated range. Where the stated range includes one or both of the limits, ranges excluding either or both of those included limits are also included in the disclosure.

As used herein, the terms “a” and “an” mean “one or more” unless specifically indicated otherwise. Additionally, “one or more” and “at least one” are used interchangeably herein. Furthermore, unless otherwise required by context, singular terms include pluralities and plural terms include the singular.

As used herein, the term “acid precipitation” refers to a harvest operation in which cell culture pH is reduced to induce precipitation of one or more cell culture impurities.

As used herein, “asparaginyl endopeptidase,” which is also referred to as “AEP,” “legumain,” and “δ-secretase,” is a lysosomal cysteine endopeptidase from the C13 peptidase family, which hydrolyzes substrates at the C-terminus of asparagine residues at mildly acidic pH (2.5<pH<4.5). AEP converts from an inactive proenzyme form to a mature, active form by auto-cleavage of terminal portions at mildly acidic pH. Zhao et al, Cell Research (2014) 24:344-358. Chinese Hamster Ovary (CHO) cell AEP is predicted to possess the following sequence, including signal peptide:

(SEQ ID NO: 3)
MCRMIWKAAVLLSLALGAGALAVGVDDPEDAGKHWVVIVAGSNGWYNYRH
QADACHAYQIIHRNGIPDEQIIVMMYDDIANSEDNPTPGIVINRPNGTDV
YAGVLKDYTGEDVTPENFLAVLRGDAEAVKGKGSGKVLRSGPQDHVFVYF
TDHGATGLLVFPNEDLHVKDLNKTIRYMYEHKMYQKMVFYIEACESGSMM
NHLPNDINVYATTAANPHESSYACYYDEERNTYLGDWYSVNWMEDSDVED
LTKETLHKQYHLVKSHTNTSHVMQYGNKSISTMKVMQFQGMKHSTSSPIS
LPPVTRLDLTPSPEVPLTILKRKLMSTNDLKQSQNLVGQIQRLLDARHVI
EKSVHKIVSLLAGFGETAERLLSERAVLMAHDCYQEAVTHFRTHCFNWHS
PTYEYALRHLYVLANLCEKPYPIDRIKMAMDKVCLSHY

AEP sequences of mouse (M. musculus), human (H. sapiens), and wild pig (S. scrofa) were aligned in Zhao et al, Cell Research (2014) 24:344-358, to create a universal numbering scheme for AEP enzymes. The sequence of CHO AEP is three residues longer due to three additional residues in the signal peptide at the N-terminus. Accordingly, CHO AEP residues N328, D308, and E314 in SEQ ID NO: 3 correspond to N325, D305, and E311 in the universal AEP numbering proposed by Zhao et al, Cell Research (2014) 24:344-358, for AEP from mouse, human, and wild pig.

As used herein, the term “antigen-binding protein” refers to a protein or polypeptide that comprises an antigen-binding region or antigen-binding portion that has affinity for another molecule to which it binds (antigen). Antigen-binding proteins include, but are not limited to, antibodies, fusion proteins, VH, VHH, VL, (s)dAb, Fv, light chain (VL-CL), Fd (VH-CH1), heavy chain, Fab, Fab′, F(ab′)2 or “r IgG” (“half antibody” consisting of a heavy chain and a light chain) or a modified antigen-binding portion of a full-length antibody, such as, e.g., a triple-chain antibody-like molecule, a heavy chain only antibody, single-chain variable fragment (scFv), di-scFv or bi(s) scFv, scFv-Fc, scFv-zipper, single-chain Fab (scFab), Fab₂, Fab₃, diabodies, single-chain diabodies, tandem diabodies (Tandabs), tandem di-scFv, tandem tri-scFv, “minibodies” exemplified by a structure which is as follows: (VH-VL-CH3)₂, (scFv-CH3)₂, ((scFv)₂-CH3+CH3), ((scFv)₂-CH3) or (scFv-CH3-scFv)₂, multibodies, such as triabodies or tetrabodies, and single domain antibodies, such as nanobodies or single variable domain antibodies comprising merely one variable region, which might be VHH, VH, or VL, that specifically binds to an antigen or target independently of other variable regions or domains.

As used herein, the term “antibody” generally refers to a tetrameric immunoglobulin protein comprising two light chain polypeptides (about 25 kDa each) and two heavy chain polypeptides (about 50-70 kDa each).

As used herein, the term “light chain” or “immunoglobulin light chain” refers to a polypeptide comprising, from amino terminus (N-terminus) to carboxyl terminus (C-terminus), a single immunoglobulin light chain variable region (VL) and a single immunoglobulin light chain constant domain (CL). The immunoglobulin light chain constant domain (CL) can be a human kappa (κ) or human lambda (λ) constant domain.

As used herein, the term “heavy chain” or “immunoglobulin heavy chain” refers to a polypeptide comprising, from amino terminus (N-terminus) to carboxyl terminus (C-terminus), a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). Heavy chains are classified as mu (μ), delta (Δ), gamma (γ), alpha (α), and epsilon (ε), and define the antibody's isotype as IgM, IgD, IgG, IgA, and IgE, respectively. The IgG-class and IgA-class antibodies are further divided into subclasses, namely, IgG1, IgG2, IgG3, and IgG4, and IgA1 and IgA2, respectively. The heavy chains in IgG, IgA, and IgD antibodies have three constant domains (CH1, CH2, and CH3), whereas the heavy chains in IgM and IgE antibodies have four constant domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (i.e. between the light and heavy chain) and between the hinge regions of the two antibody heavy chains.

Variable regions of immunoglobulin chains generally exhibit the same overall structure, comprising relatively conserved framework regions (FR) joined by three hypervariable regions, more often called “complementarity determining regions” or CDRs. The CDRs from the two chains of each heavy chain and light chain pair typically are aligned by the framework regions to form a structure that binds specifically to a specific epitope on the target protein. From N-terminus to C-terminus, naturally-occurring light and heavy chain variable regions both typically conform with the following order of these elements: FR1, CDR1, FR2, CDR2, FR3, CDR3, and FR4. A numbering system has been devised for assigning numbers to amino acids that occupy positions in each of these domains. This numbering system is defined in Kabat Sequences of Proteins of Immunological Interest (1987 and 1991, NIH, Bethesda, MD), or Chothia & Lesk, 1987, J. Mol. Biol. 196:901-917; Chothia et al., 1989, Nature 342:878-883. The CDRs and FRs of a given antibody may be identified using this system. Other numbering systems for the amino acids in immunoglobulin chains include IMGT® (the international ImMunoGeneTics information system; Lefranc et al., Dev. Comp. Immunol. 29:185-203; 2005) and AHo (Honegger and Pluckthun, J. Mol. Biol. 309(3):657-670; 2001).

Papain digestion of antibodies produces two identical antigen-binding proteins, called “Fab” fragments, each with a single antigen-binding site, and a residual “Fc” fragment which contains all but the first domain of the immunoglobulin heavy chain constant region. The Fab fragment contains the variable domains from the light and heavy chains, as well as the constant domain of the light chain and the first constant domain (CH1) of the heavy chain. Thus, a “Fab fragment” is comprised of one immunoglobulin light chain (light chain variable region (VL) and constant region (CL)) and the CH1 domain and variable region (VH) of one immunoglobulin heavy chain. The heavy chain of a Fab molecule cannot form a disulfide bond with another heavy chain molecule. The “Fd fragment” comprises the VH and CH1 domains from an immunoglobulin heavy chain. The Fd fragment represents the heavy chain component of the Fab fragment.

The “Fc fragment” or “Fc region” of an immunoglobulin generally comprises two constant domains, a CH2 domain and a CH3 domain, and optionally comprises a CH4 domain. The Fc region may be an Fc region from an IgG1, IgG2, IgG3, or IgG4 immunoglobulin. In some embodiments, the Fc region comprises CH2 and CH3 domains from a human IgG1 or human IgG2 immunoglobulin. The Fc region may retain effector function, such as C1q binding, complement dependent cytotoxicity (CDC), Fc receptor binding, antibody-dependent cell-mediated cytotoxicity (ADCC), and phagocytosis. In other embodiments, the Fc region may be modified to reduce or eliminate effector function.

A “F(ab′)₂ fragment” is a bivalent fragment including two Fab′ fragments linked by a disulfide bridge between the heavy chains at the hinge region.

The “Fv” fragment is the minimum fragment that contains a complete antigen recognition and binding site from an antibody. This fragment consists of a dimer of one immunoglobulin heavy chain variable region (VH) and one immunoglobulin light chain variable region (VL) in tight, non-covalent association. It is in this configuration that the three CDRs of each variable region interact to define an antigen binding site on the surface of the VH-VL dimer. A single light chain or heavy chain variable region (or half of an Fv fragment comprising only three CDRs specific for an antigen) has the ability to recognize and bind antigen, although at a lower affinity than the entire binding site comprising both VH and VL.

A “single-chain variable fragment” or “scFv fragment” comprises the VH and VL regions of an antibody, wherein these regions are present in a single polypeptide chain, and optionally comprising a peptide linker between the VH and VL regions that enables the Fv to form the desired structure for antigen binding (see e.g., Bird et al., Science, Vol. 242:423-426, 1988; and Huston et al., Proc. Natl. Acad. Sci. USA, Vol. 85:5879-5883, 1988).

A “nanobody” is the heavy chain variable region of a heavy-chain antibody. Such variable domains are the smallest fully functional antigen-binding fragment of such heavy-chain antibodies with a molecular mass of only 15 kDa. See Cortez-Retamozo et al., Cancer Research 64:2853-57, 2004. Functional heavy-chain antibodies devoid of light chains are naturally occurring in certain species of animals, such as nurse sharks, wobbegong sharks, and Camelidae, such as camels, dromedaries, alpacas and llamas. The antigen-binding site is reduced to a single domain, the VHH domain, in these animals. These antibodies form antigen-binding regions using only heavy chain variable region, i.e., these functional antibodies are homodimers of heavy chains only having the structure H₂L₂ (referred to as “heavy-chain antibodies” or “HCAbs”). Camelized VHH reportedly recombines with IgG2 and IgG3 constant regions that contain hinge, CH2, and CH3 domains and lack a CH1 domain. Camelized VHH domains have been found to bind to antigen with high affinity (Desmyter et al., J. Biol. Chem., Vol. 276:26285-90, 2001) and possess high stability in solution (Ewert et al., Biochemistry, Vol. 41:3628-36, 2002). Methods for generating antibodies having camelized heavy chains are described in, for example, U.S. Patent Publication Nos. 2005/0136049 and 2005/0037421. Alternative scaffolds can be made from human variable-like domains that more closely match the shark V-NAR scaffold and may provide a framework for a long penetrating loop structure.

As used herein, the term “heavy chain-only antibody” refers to an immunoglobulin protein consisting of two heavy chain polypeptides (such as, e.g., heavy chain polypeptides that are about 50-70 kDa each). A “heavy chain-only antibody” lacks the two light chain polypeptides found in a conventional antibody. Heavy-chain antibodies constitute about-one fourth of the IgG antibodies produced by the camelids, e.g., camels and llamas (Hamers-Casterman C., et al. Nature. 363, 446-448 (1993)). These molecules are formed by two heavy chains but are devoid of light chains. As a consequence, the variable antigen binding part is referred to as the VHH domain, and it represents the smallest naturally occurring, intact, antigen-binding site, being only around 120 amino acids in length (Desmyter, A., et al. J. Biol. Chem. 276, 26285-26290 (2001)). Heavy chain antibodies with a high specificity and affinity can be generated against a variety of antigens through immunization (van der Linden, R. H., et al. Biochim. Biophys. Acta. 1431, 37-46 (1999)), and the VHH portion can be readily cloned and expressed in yeast (Frenken, L. G. J., et al. J. Biotechnol. 78, 11-21 (2000)). Their levels of expression, solubility and stability are significantly higher than those of classical F(ab) or Fv fragments (Ghahroudi, M. A. et al. FEBS Lett. 414, 521-526 (1997)). Sharks have also been shown to have a single VH-like domain in their antibodies, termed VNAR. (Nuttall et al. Eur. J. Biochem. 270, 3543-3554 (2003); Nuttall et al. Function and Bioinformatics 55, 187-197 (2004); Dooley et al., Molecular Immunology 40, 25-33 (2003).)

In some embodiments, a “heavy chain-only antibody” is a dimeric antibody comprising a VH antigen-binding domain and the CH2 and CH3 constant domains, in the absence of the CH1 domain. In some embodiments, a heavy chain-only antibody is composed of a variable region antigen-binding domain composed of framework 1, CDR1, framework 2, CDR2, framework 3, CDR3, and framework 4. In some embodiments, a heavy chain-only antibody is composed of an antigen-binding domain, at least part of a hinge region, and CH2 and CH3 domains. In some embodiments, a heavy chain-only antibody is composed of an antigen-binding domain, at least part of a hinge region, and a CH2 domain. In some embodiments, a heavy chain-only antibody is composed of an antigen-binding domain, at least part of a hinge region, and a CH3 domain. Heavy chain-only antibodies in which the CH2 and/or CH3 domain is truncated are also included herein. The heavy chain-only antibodies described herein may belong to the IgG subclass, but heavy chain-only antibodies belonging to other subclasses, such as IgM, IgA, IgD and IgE subclass, are also included herein. In some embodiments, a heavy chain-only antibody may belong to the IgG1, IgG2, IgG3, or IgG4 subtype, e.g., the IgG1 or IgG4 subtype. In some embodiments, a heavy chain antibody-only is of the IgG1 or IgG4 subtype, wherein one or more of the CH domains is modified to alter an effector function of the antibody. In some embodiments, a heavy chain-only antibody is of the IgG4 subtype, wherein one or more of the CH domains is modified to alter an effector function of the antibody. In some embodiments, a heavy chain-only antibody is of the IgG1 subtype, wherein one or more of the CH domains is modified to alter an effector function of the antibody. Modifications of CH domains that alter effector function are further described herein. Non-limiting examples of heavy-chain-only antibodies are described, for example, in WO2018/039180, the disclosure of which is incorporated herein by reference herein in its entirety.

As used herein, the term “three-chain antibody like molecule” or “TCA” refers to an antibody-like molecule comprising, consisting essentially of, or consisting of three polypeptide subunits, two of which comprise, consist essentially of, or consist of one heavy and one light chain of a monoclonal antibody, or antigen-binding fragments of such antibody chains, comprising an antigen-binding region and at least one CH domain. This heavy chain/light chain pair has binding specificity for a first antigen. The third polypeptide subunit comprises, consists essentially of, or consists of a heavy-chain only antibody comprising an Fc portion comprising CH2 and/or CH3 and/or CH4 domains, in the absence of a CH1 domain, and one or more antigen binding domains (such as, e.g., two antigen binding domains) that binds an epitope of a second antigen or a different epitope of the first antigen, where such binding domain is derived from or has sequence identity with the variable region of an antibody heavy or light chain. Parts of such variable region may be encoded by V_{H} and/or V_{L} gene segments, D and J_{H} gene segments, or J_{L} gene segments. The variable region may be encoded by rearranged V_{H}DJ_{H}, V_{L}D_{JH}, V_{H}J_{L}, or V_{L}J_{L} gene segments.

As used herein, the term “bioreactor” means any vessel useful for the growth of a cell culture (e.g., a mammalian cell culture or a bacterial cell culture). “Bioreactor” encompasses the term “fermenter” (i.e., a vessel useful for the growth of a bacterial cell culture, which typically contains a more rigorous agitator and increased gas flow relative to a vessel used for the growth of a mammalian cell culture) herein. Non-limiting examples of bioreactors include stirred tank, airlift, fiber, microfiber, hollow fiber, ceramic matrix, fluidized bed, fixed bed, and/or spouted bed bioreactors. In some embodiments, an example bioreactor can perform one or more (e.g., one, two, three, all) of the following steps: feeding of nutrients and/or carbon sources, injection of suitable gas (such as, e.g., oxygen), inlet and outlet flow of fermentation or cell culture medium (e.g., perfusion of fresh cell culture medium in and removal of spent cell culture medium), separation of gas and liquid phases, maintenance of temperature, maintenance of oxygen and CO₂ levels, maintenance of pH level, agitation (e.g., stirring), and/or cleaning/sterilizing. Unless otherwise indicated by context, a bioreactor can be suitable for batch, semi fed-batch, fed-batch, perfusion, and/or continuous fermentation processes. Any suitable bioreactor diameter can be used. Unless otherwise indicated by context, in some embodiments, the bioreactor can have a volume between 100 mL and 50,000 L. Unless otherwise indicated, a bioreactor can be of any size so long as it is useful for the culturing of cells; typically, a bioreactor is sized appropriate to the volume of cell culture being grown inside of it. In non-limiting embodiments and unless otherwise indicated by context, a bioreactor may be at least 1 liter (L) or may be 2, 5, 10, 50, 100, 200, 250, 500, 1,000, 1500, 2000, 2,500, 5,000, 8,000, 10,000, 12,000, 20,000 liters or more, or any volume in between. The internal conditions of the bioreactor, including, but not limited to, pH, dissolved oxygen (DO), and temperature, can be controlled during the culturing period. Those of ordinary skill in the art will be aware of, and will be able to select, suitable bioreactors for use in methods disclosed herein based on the relevant considerations.

As used herein, the term “cell culture” or “culture” refers to the growth and propagation of cells outside of a multicellular organism or tissue. Suitable culture conditions for mammalian and bacterial cells are known in the art. (See, e.g., Animal cell culture: A Practical Approach, D. Rickwood, ed., Oxford University Press, New York (1992).) Mammalian cells may be cultured in suspension or while attached to a solid substrate. In some embodiments, fluidized bed bioreactors, hollow fiber bioreactors, roller bottles, shake flasks, and/or stirred tank bioreactors, with or without microcarriers, may be used for cell culture. In some embodiments, 500 L to 2000 L bioreactors are used for cell culture (e.g., as part of a seed train). In some embodiments, 1000 L to 2000 L bioreactors are used for cell culture (e.g., as part of a seed train).

As used herein, the term “cell culturing medium” (also referred to as “media,” “culture medium,” “cell culture media,” “tissue culture media,” and the like) refers to any nutrient solution used for growing cells, e.g., bacterial or mammalian cells. Cell culturing medium generally provides one or more of the following components: an energy source (e.g., in the form of a carbohydrate, such as, e.g., glucose); one or more essential amino acids (e.g., all essential amino acids; the twenty basic amino acids plus cysteine); vitamins and/or other organic compounds typically required at low concentrations; lipids or free fatty acids; and trace elements, such as, e.g., inorganic compounds or naturally occurring elements that are typically required at very low concentrations, such as, e.g., concentrations in the micromolar range. As used herein, cell culturing medium encompasses nutrient solutions that are typically employed in and/or are known for use with any cell culture process, including, but not limited to, batch, extended batch, fed-batch, intensified, and/or perfusion or continuous culturing of cells.

As used herein, the term “cell density” refers to the number of cells in a given volume of culture medium. “Viable cell density” refers to the number of live cells in a given volume of culture medium, as determined by standard viability assays (such as, e.g., a trypan blue dye exclusion method) and may be measured at any point during a specific phase of a cell culture process. As used herein, the term “packed cell volume” (PCV), also referred to as “percent packed cell volume” (% PCV), is the ratio of the volume occupied by the cells, to the total volume of cell culture, expressed as a percentage (see Stettler, et al., (2006) Biotechnol Bioeng. December 20:95(6): 1228-33). Packed cell volume is a function of cell density and cell diameter; increases in packed cell volume could arise from increases in cell density or cell diameter or both. Packed cell volume is a measure of the solid content in the cell culture. Since host cells vary in size and cell cultures also contain dead and dying cells and other cellular debris, packed cell volume can describe with a greater degree of accuracy the solid content within a cell culture.

As used herein, the term “expression vector” or “expression construct” refers to a recombinant DNA molecule containing a desired coding sequence and appropriate nucleic acid control sequences necessary for the expression of the operably linked coding sequence in a particular host cell, e.g. a mammalian host cell. Vectors can include viral vectors, non-episomal mammalian vectors, plasmids, and other non-viral vectors. An expression vector can include sequences that affect or control transcription, translation, and, if introns are present, affect RNA splicing of a coding region operably linked thereto. “Operably linked” means that the components to which the term is applied are in a relationship that allows them to carry out their inherent functions. For example, a control sequence, e.g., a promoter, in a vector that is “operably linked” to a protein coding sequence are arranged such that normal activity of the control sequence leads to transcription of the protein coding sequence resulting in recombinant expression of the encoded protein.

As used herein, “fed-batch culture” refers to a form of suspension culture, specifically a method of culturing cells in which additional components are provided to the culture at a time or times subsequent to the beginning of the culture process. The provided components typically comprise nutritional supplements for the cells which have been depleted during the culturing process. Additionally or alternatively, the additional components may include supplementary components (such as, e.g., a cell-cycle inhibitory compound). In some embodiments, fed-batch cell culture medium formulations may be richer or more concentrated than basal cell culture medium formulations, which contain components essential for cell survival and growth and are typically used to initiate a cell culture. A fed-batch culture may be stopped at some point, and the cells and/or components in the medium may be harvested and optionally purified.

As used herein, a “fusion protein” is a protein that contains at least one polypeptide fused or linked to a heterologous polypeptide. Typically, a fusion protein is expressed from a fusion gene in which a nucleotide sequence encoding a polypeptide sequence from one protein is appended in frame with, and optionally separated by a linker from, a nucleotide sequence encoding a polypeptide sequence from a different protein. The fusion gene can then be expressed by a recombinant host cell to produce the fusion protein. The fusion protein may comprise a fragment from an immunoglobulin protein, such as an Fc region, fused or linked to a ligand polypeptide, a receptor polypeptide, a hormone, cytokine, growth factor, an enzyme, or other polypeptide that is not a component of an immunoglobulin.

As used herein, a “growth phase” of a cell culture refers to the period of exponential cell growth (i.e. the log phase) where cells are generally rapidly dividing.

As used herein, the term “harvested cell culture fluid” refers to a solution which has been processed by one or more operations to separate cells, cell debris, or other large particulates from the recombinant protein. Such operations, as described above, include, but are not limited to, cooling, flocculation, acidification, centrifugation, neutralization, acoustic wave separation, and various forms of filtration (e.g., depth filtration, microfiltration, ultrafiltration, tangential flow filtration, and alternating tangential flow filtration). Harvested cell culture fluid includes cell culture lysates as well as cell culture supernatants. The harvested cell culture fluid may be further clarified to remove fine particulate matter and soluble aggregates by filtration with a membrane having a pore size between about 0.1 μm and about 0.5 μm, such as, e.g., a membrane having a pore size of about 0.22 μm.

As used herein, a “host cell” refers to a cell that has been transformed, or is capable of being transformed, with a nucleic acid and thereby expresses a gene of interest. The term includes the progeny of the parent cell, whether or not the progeny is identical in morphology or in genetic make-up to the original parent cell, so long as the gene of interest is present. A host cell that comprises a nucleic acid encoding a recombinant protein, e.g., operably linked to at least one expression control sequence (e.g. promoter or enhancer), is a “recombinant host cell.” A host cell, when cultured under appropriate conditions, may synthesize a recombinant protein that can subsequently be collected from the culture medium (if the host cell secretes it into the medium) or directly from the host cell producing it (if it is not secreted).

As used herein, “low molecular weight” or “LMW” species of a recombinant protein of interest refer to fragments, truncated forms, or other incomplete variants of the recombinant protein that have a molecular weight less than the molecular weight of the intact, fully assembled form of the recombinant protein. LMW species can include, but are not limited to, proteolytic fragments, truncated forms resulting from cellular expression of mRNA splice variants, and single component polypeptides in the case of multi-polypeptide chain proteins (e.g. light chain or heavy chain only species when the recombinant protein is an antibody).

As used herein, a “perfusion” cell culture medium refers to a cell culture medium that is typically used in cell cultures that are maintained by perfusion or continuous culture methods and is sufficiently complete to support the cell culture during this process. In some embodiments, perfusion cell culture medium formulations may be richer or more concentrated than base cell culture medium formulations to accommodate the method used to remove the spent medium. In some embodiments, perfusion cell culture medium may be used during both the growth and production phases.

As used herein, the term “polypeptide” refers to a polymer of amino acids comprising at least 50 amino acids, such as, e.g., at least 100 amino acids.

As used herein, a “production” cell culture medium refers to a cell culture medium that is typically used in a cell culture during the transition when exponential growth is ending and protein production takes over (i.e., “transition” and/or “product” phases) and is sufficiently complete to maintain a desired cell density, viability, and/or product titer during this phase. A production cell culture medium may be the same as or different than the cell culture medium used during the exponential growth phase of the cell culture.

As used herein, a “production phase” of a cell culture refers to the period of time during which logarithmic cell growth has ended and recombinant protein production is predominant.

As used herein, the term “protease inhibitor” refers to a molecule that at least partially inhibits the function of one or more protein-based enzymes that cleave other proteins. In some embodiments of the present disclosure, an exogenous protease inhibitor is added to a cell culture to inhibit cleavage of the recombinant protein of interest. In some embodiments, the exogenous protease inhibitor is a commercially available protease inhibitor cocktail intended to increase secreted protein stability, such as, e.g., E-64 protease inhibitor, TCM ProteaseArrest™ Protease Inhibitor Cocktail (G-Biosciences), Protease Inhibitor Cocktail I (R&D Systems), Halt™ Protease Inhibitor Cocktail (Thermo Scientific), or Protease Inhibitor Cocktail (Promega or Sigma-Aldrich). In some embodiments, the exogenous protease inhibitor is an AEP inhibitor. In some embodiments, the AEP inhibitor is AENK, which is commercially available, e.g., from Sigma-Aldrich. In some embodiments, the AEP inhibitor is 8-secretase inhibitor 11 (7-morpholin-4-yl-benzo[1,2,5]oxadiazol-4-ylamine). In some embodiments, the AEP inhibitor is 7-morpholinobenzo[c][1,2,5]oxadiazol-4-amine. In some embodiments, the AEP inhibitor is a substituted 3,7-dihydropurine-2,6-dione derivative described in U.S. Patent Application Publication No. 2017/0166569.

As used herein, the term “recombinant protein” refers to a heterologous protein produced by a host cell transfected with a nucleic acid encoding the protein when the host cell is cultivated in cell culture.

As used herein, the term “unit operation” refers to a functional step that is performed as part of a process of purifying a recombinant protein of interest. Unit operations can be designed to achieve a single objective or multiple objectives, such as capture and virus inactivating steps. Unit operations can also include holding or storing steps between processing steps.

### Host Cells

Cell lines (also referred to as “cells” or “host cells”) used in the present disclosure are genetically engineered to express a recombinant protein of commercial or scientific interest. Cells may be suitable for adherent, monolayer, and/or suspension culture, transfection, and expression of recombinant proteins, such as, e.g., antibodies. The cells can be used, for example, with batch, fed batch, and perfusion or continuous culture methods. Such cells are typically cell lines obtained or derived from mammals and are able to grow and survive when placed in either monolayer culture or suspension culture in medium containing appropriate nutrients and/or other factors, such as those described herein. The cells are typically selected that can express and secrete proteins, or that can be molecularly engineered to express and secrete, large quantities of a particular protein, more particularly, a glycoprotein of interest, into the culture medium. The selection of an appropriate host cell for expressing a recombinant protein will depend upon various factors, such as desired expression levels, polypeptide modifications that are desirable or necessary for activity (such as glycosylation or phosphorylation) and case of folding into a biologically active molecule. In some embodiments of the methods of the present disclosure, the host cell is a mammalian host cell.

Cell lines are typically derived from a lineage arising from a primary culture that can be maintained in culture for an unlimited time. The cells can contain introduced, e.g., via transformation, transfection, infection, or injection, expression vectors (constructs), such as plasmids and the like, that harbor coding sequences, or portions thereof, encoding the proteins for expression and production in the culturing process. Such expression vectors contain the necessary elements for the transcription and translation of the inserted coding sequence. Methods which are well known to and practiced by those skilled in the art can be used to construct expression vectors containing sequences encoding the produced proteins and polypeptides, as well as the appropriate transcriptional and translational control elements. These methods include, but are not limited to, in vitro recombinant DNA techniques, synthetic techniques, and in vivo genetic recombination. Such techniques are described in J. Sambrook et al., 2012, Molecular Cloning, A Laboratory Manual, 4^{th} edition Cold Spring Harbor Press, Plainview, N.Y. or any of the previous editions; F. M. Ausubel et al., 2013, Current Protocols in Molecular Biology, John Wiley & Sons, New York, N. Y, or any of the previous editions; Kaufman, R. J., Large Scale Mammalian Cell Culture, 1990, all of which are incorporated herein for any purpose.

Suitable host cells include, but are not limited to, those that are commercially available, for example, from culture collections such as the DSMZ (Deutsche Sammlung von Mikroorganismen and Zellkulturen GmbH, Braunschweig, Germany) or the American Type Culture Collection (ATCC).

In some embodiments, the host cells are selected from CHO cells. CHO cells, including CHOK1 cells (ATCC CCL61), are widely used to produce complex recombinant proteins. In some embodiments, the dihydrofolate reductase (DHFR)-deficient mutant cell lines (Urlaub et al., 1980, Proc Natl Acad Sci USA 77: 4216-4220), DXB11 and DG-44, are desirable CHO host cell lines because the efficient DHFR selectable and amplifiable gene expression system allows high level recombinant protein expression in these cell lines (Kaufman R. J., 1990, Meth Enzymol 185:537-566). Also included are the glutamine synthase (GS)-knockout CHOKISV cell lines, making use of glutamine synthetase (GS)-based methionine sulfoximine (MSX) selection. Other suitable CHO host cells for use in a biomanufacturing process disclosed herein include, but are not limited to, the following (ECACC accession numbers in parenthesis): CHO (85050302); CHO (PROTEIN FREE) (00102307); CHO-K1 (85051005); CHO-K1/SF (93061607); CHO/dhFr-(94060607); CHO/dhFr-AC-free (05011002); and RR-CHOKI (92052129).

To generate host cell lines (e.g., mammalian cell lines) engineered to express a recombinant protein of interest, one or more nucleic acids encoding the recombinant protein (or components thereof in the case of multi-chain proteins) is initially inserted into one or more expression vectors. Nucleic acid control sequences useful in expression vectors for expression in mammalian cells include promoters, enhancers, and termination and polyadenylation signals. A secretory signal peptide sequence can also, optionally, be encoded by the expression vector, operably linked to the coding sequence of interest, so that the expressed protein can be secreted by the recombinant host cell, for more facile isolation of the recombinant protein from the cell, if desired. Vectors may also include one or more selectable marker genes to facilitate selection of host cells into which the vectors have been introduced. In some embodiments, vectors are used that employ protein-fragment complementation assays using protein reporters, such as dihydrofolate reductase (see, for example, U.S. Pat. No. 6,270,964). Suitable mammalian expression vectors are known in the art and are also commercially available.

Typically, vectors used in any of the host cells will contain sequences for plasmid maintenance and for cloning and expression of exogenous nucleotide sequences. Such sequences will typically include one or more of the following nucleotide sequences: a promoter, one or more enhancer sequences, an origin of replication, transcriptional and translational control sequences, a transcriptional termination sequence, a complete intron sequence containing a donor and acceptor splice site, a native or heterologous signal peptide sequence (leader sequence or signal peptide) for polypeptide secretion, a ribosome binding site, a polyadenylation sequence, a polylinker region for inserting the polynucleotide encoding the polypeptide to be expressed, and a selectable marker element. Vectors may be constructed from a starting vector such as a commercially available vector, and additional elements may be individually obtained and ligated into the vector.

### Culture Methods

Various culture methods may be used to produce a recombinant protein of interest, including, but not limited to, batch culture, fed-batch culture, perfusion culture, and intensified cell culture.

Batch culture is a discontinuous method where cells are grown in a fixed volume of culture media for a short period of time followed by a full harvest. Cultures grown using the batch method experience an increase in cell density until a maximum cell density is reached, followed by a decline in viable cell density as the media components are consumed and levels of metabolic by-products (such as lactate and ammonia) accumulate. Harvest typically occurs at the point when the maximum cell density is achieved (e.g., 5×10⁶ cells/mL or greater, depending on media formulation, cell line, etc.). The batch process is the simplest culture method; however, viable cell density is limited by nutrient availability and once the cells are at maximum density, the culture declines and production decreases. There is no ability to extend a production phase in batch culture because the accumulation of waste products and nutrient depletion rapidly lead to culture decline, typically around 3 to 7 days.

Fed-batch culture improves on the batch process by providing bolus or continuous media feeds to replenish those media components that have been consumed. Since fed-batch cultures receive additional nutrients throughout the run, they have the potential to achieve higher cell densities (>10 to 30×10⁶ cells/mL, depending on media formulation, cell line, etc.) and increased product titers, when compared to the batch method. Unlike the batch process, a biphasic culture can be created and sustained by manipulating feeding strategies and media formulations to distinguish the period of cell proliferation to achieve a desired cell density (the growth phase) from the period of suspended or slow cell growth (the production phase). As such, fed-batch cultures have the potential to achieve higher product titers compared to batch cultures. Typically, a batch method is used during the growth phase and a fed-batch method used during the production phase, but a fed-batch feeding strategy can be used throughout the entire process. However, unlike the batch process, bioreactor volume is a limiting factor which limits the amount of feed. Also, as with the batch method, metabolic by-product accumulation will lead to culture decline, which limits the duration of the production phase, often around 10 to 21 days. Fed-batch cultures are discontinuous, and harvest typically occurs when metabolic by-product levels or culture viability reach predetermined levels. When compared to a batch culture, in which no feeding occurs, a fed-batch culture can produce greater amounts of recombinant protein. (See, e.g., U.S. Pat. No. 5,672,502.)

Perfusion methods offer potential improvements over the batch and fed-batch methods by adding fresh media and simultaneously removing spent media during culture. Typical perfusion cultures begin with a batch culture start-up lasting for a day or two followed by continuous, stepwise, and/or intermittent addition of fresh feed media to the culture and simultaneous removal of spent media with the retention of cells and additional high molecular weight compounds such as proteins (based on the filter molecular weight cutoff) throughout the growth and production phases of the culture. Various methods, such as sedimentation, centrifugation, or filtration, can be used to remove spent media, while maintaining cell density. Non-limiting examples of filtration methods include alternating tangential flow filtration and recirculating tangential flow. Alternating tangential flow is maintained by pumping medium through hollow-fiber filter modules. See e.g. U.S. Pat. No. 6,544,424; Furcy, 2002, Gen. Eng. News. 22 (7):62-63.

Perfusion can be continuous, stepwise, intermittent, or a combination of any or all of any of these. Perfusion rates can be less than a working volume to many working volumes per day. The cells are retained in the culture, and the spent medium that is removed is substantially free of cells or has significantly fewer cells than the culture. Recombinant proteins expressed by the cell culture can also be retained in the culture.

Typical large scale commercial cell culture strategies strive to reach high cell densities, such as, e.g., 30-90(+)×10⁶ cells/mL, where almost a third to over one-half of the reactor volume is biomass. With perfusion culture, extreme cell densities of >1×10⁸ cells/mL have been achieved. A potential advantage of the perfusion process is that the production culture can be maintained for longer periods than batch or fed-batch culture methods. However, increased media preparation, use, storage, and disposal are necessary to support a long-term perfusion culture, particularly for a culture with high cell density, which also needs even more nutrients. All of this can increase production costs compared to batch and fed-batch methods. In addition, higher cell densities can cause problems during production, such as, e.g., maintaining dissolved oxygen levels and problems with increased gassing, including supplying more oxygen and removing more carbon dioxide, which could result in more foaming and the need for alterations to antifoam strategies; as well as during harvest and downstream processing where the efforts required to remove the excessive cell material can result in loss of product, negating the benefit of increased titer due to increased cell mass.

Suitable culture conditions, including temperature, dissolved oxygen content, agitation rate, and the like, for mammalian cells are known in the art and may vary by the phase or stage of the cell culture. In some embodiments, the methods disclosed herein further comprise taking samples during the cell culture processes, evaluating the samples to quantitatively and/or qualitatively monitor characteristics of the recombinant protein and/or the cell culture process. In some embodiments, the samples are quantitatively and/or qualitatively monitored using process analytical techniques. For examples, dissolved oxygen levels may be monitored during the cell culture processes using methods known in the art, such as, e.g., a basic chemical analysis method (titration method), an electrochemical analysis method (diaphragm electrode method), and a photochemical analysis method (fluorescence method).

During recombinant protein production, it is desirable to have a controlled system where cells are grown for a desired time or to a desired density and then the physiological state of the cells is switched to a growth-limited or arrested, high productivity state where the cells use energy and substrates to produce the recombinant protein in favor of increasing cell density. For commercial scale cell culture and the manufacture of biological therapeutics, the ability to limit or arrest cell growth and being able to maintain the cells in a growth-limited or arrested state during the production phase is very desirable. Such methods include, for example, temperature shifts, use of chemical inducers of protein production, nutrient limitation or starvation and cell cycle inhibitors, either alone or in combination. Illustratively, a typical cell culture undergoes a growth phase, this is a period of exponential growth where cell density is increased. During the growth phase, cells are cultured in a cell culture medium containing the necessary nutrients and additives under conditions (generally at about a temperature of 25°-40° ° C., in a humidified, controlled atmosphere) such that optimal growth is achieved for the particular cell line. Cells are typically maintained in the growth phase for a period of between one and eight days, e.g., between three to seven days, e.g., seven days. The length of the growth phase for a particular cell line can be determined by a person of ordinary skill in the art and will generally be the period of time sufficient to allow the particular cells to reproduce to a viable cell density within a range of about 20%-80% of the maximal possible viable cell density if the culture was maintained under the growth conditions. The growth phase is followed by a transition phase when exponential cell growth is slowing and protein production starts to increase. This marks the start of the stationary phase, a production phase, where cell density typically levels off and product titer increases. During the production phase, the medium is generally supplemented to support continued recombinant protein production.

In certain embodiments of the methods of the present disclosure, the culture conditions may be adjusted to facilitate the transition from the growth phase of the cell culture to the production phase. For instance, a growth phase of the cell culture may occur at a higher temperature than a production phase of the cell culture. In some embodiments, a growth phase may occur at a first temperature from about 35° ° C. to about 38° C., and a production phase may occur at a second temperature from about 29° ° C. to about 37° C., optionally from about 30° C. to about 36° C. or from about 30° ° C. to about 34° C. In one embodiment, a shift in temperature from about 35° C. to about 37° ° C. to a temperature of about 31° ° C. to about 33° ° C. may be employed to facilitate the transition from the growth phase of the culture to the production phase. Chemical inducers of protein production, such as, for example, caffeine, butyrate, and hexamethylene bisacetamide (HMBA), may be added at the same time as, before, and/or after a temperature shift, or in place of a temperature shift. If inducers are added after a temperature shift, they can be added from one hour to five days after the temperature shift, optionally from one to two days after the temperature shift.

Additionally, any cell culture media capable of supporting growth of the appropriate host cell in culture can be used. Typically, the cell culture medium contains a buffer, salts, energy source, amino acids, vitamins and trace essential elements. Cell culture media, which may be further supplemented with other components to maximize cell growth, cell viability, and/or recombinant protein production in a particular cultured host cell, are commercially available and include RPMI-1640 Medium, RPMI-1641 Medium, Dulbecco's Modified Eagle's Medium (DMEM), Minimum Essential Medium Eagle, F-12K Medium, Ham's F12 Medium, Iscove's Modified Dulbecco's Medium, McCoy's 5A Medium, Leibovitz's L-15 Medium, and serum-free media such as EX-CELL™ 300 Series, among others, which can be obtained from the American Type Culture Collection or SAFC Biosciences, as well as other vendors. Cell culture media can be serum-free, protein-free, growth factor-free, and/or peptone-free media. Cell culture media may also be enriched by the addition of nutrients or other supplements, which may be used at greater than usual, recommended concentrations. In certain embodiments, the culture medium used in the methods of the present disclosure is a chemically defined medium, which refers to a cell culture medium in which all of the components have known chemical structures and concentrations. Chemically defined media are typically serum-free and do not contain hydrolysates or animal-derived components.

Various media formulations can be used during the life of the culture, for example, to facilitate the transition from one stage (e.g., the growth stage or phase) to another (e.g., the production stage or phase) and/or to optimize conditions during cell culture (e.g. concentrated media provided during a perfusion culture). A growth medium formulation can be used to promote cell growth and minimize protein expression. A production medium formulation can be used to promote production of the recombinant protein of interest and maintenance of the cells, with minimal new cell growth. A feed medium is typically a cell culture medium containing more concentrated components such as nutrients and amino acids, which are consumed during the course of the production phase of the cell culture. Feed medium may be used to supplement and maintain an active culture, particularly a culture operated in fed batch, semi-perfusion, or perfusion mode. Such a concentrated feed medium can contain most of the components of the cell culture medium at, for example, about 5×, 6×, 7×, 8×, 9×, 10×, 12×, 14×, 16×, 20×, 30×, 50×, 100×, 200×, 400×, 600×, 800×, or even about 1000× of their normal amount.

In some embodiments of the methods of the present disclosure, the mammalian cell is cultured for a defined period of time during which the recombinant protein is expressed and secreted by the mammalian cell. This period of time (i.e. the duration of the production phase of the cell culture) is at least 3 days, at least 7 days, at least 10 days, or at least 15 days. In certain embodiments, the duration of the production phase of the cell culture is about 7 days to about 28 days, about 10 days to about 30 days, about 7 days to about 14 days, about 10 days to about 18 days, about 3 days to about 15 days, about 5 days to about 8 days, about 12 days to about 15 days, about 12 days to about 18 days, or about 15 days to about 21 days. In some embodiments, the duration of the production phase of the cell culture is 7 days, 8 days, 9 days, 12 days, 15 days, 18 days, or 21 days.

In some embodiments of the methods of the present disclosure, the biomanufacturing process comprises a production phase with a viable cell density of at least 100×10⁵ cells/mL, for example between about 100×10⁵ cells/mL and about 10×10⁷ cells/mL, between about 250×10⁵ cells/mL and about 900×10⁵ cells/mL, between about 300×10⁵ cells/mL and 800×10⁵ cells/mL, or between about 450×10⁵ cells/mL and 650×10⁵ cells/mL. Cell density may be measured using a hemacytometer, a Coulter counter, or an automated cell analyzer (e.g. Cedex automated cell counter). Viable cell density may be determined by staining a culture sample with Trypan blue, which is taken up only by dead cells. Viable cell density is then determined by counting the total number of cells, dividing the number of stained cells by the total number of cells, and taking the reciprocal.

In some embodiments of the methods of the present disclosure, the biomanufacturing process comprises a production phase with a packed cell volume less than or equal to 35%. In some embodiments, the packed cell volume is less than or equal to 30%.

### Purification Processes

The expressed recombinant proteins may be secreted into the culture medium from which they can be recovered and/or collected. Harvest operations comprising an acid precipitation may be combined with additional harvest strategies, including centrifugation, such as disk-stack centrifugation, intermittent discharge centrifugation, or continuous solid discharge centrifugation; filtration, including tangential flow filtration, microfiltration, ultrafiltration, and depth filtration; precipitation/sedimentation methods, such as flocculation; and chromatography media-based separations.

Beyond a harvest operation comprising an acid precipitation, the present disclosure comprehends methods involving all known post-harvest recovery technologies, such as, e.g., protein A purification of immunoglobulin and immunoglobulin-like biologics, as well as chromatography-based separations and polishing steps that include column and alternative modes of chromatographic separations by ion exchange chromatography (IEX), including anion exchange chromatography (AEX) and/or cation exchange chromatography (CEX), hydrophobic interaction chromatography (HIC), mixed modal or multimodal chromatography (MM), hydroxyapatite chromatography (HA), reverse-phase chromatography, size exclusion chromatography (SEC), gel filtration, or any other known form of chromatographic separation of biological and/or biochemical substances.

In some embodiments of the methods of the present disclosure, recombinant protein recovered from the host cells or cell culture medium may be further purified or partially purified to remove cell culture media components, host cell proteins, or nucleic acids, or other process or product-related impurities by one or more unit operations. One of ordinary skill in the art can select the appropriate unit operation(s) for further purification of a recombinant protein based on the characteristics of the recombinant protein to be purified, the characteristics of host cell from which the recombinant protein is expressed, and the composition of the culture medium in which the host cells were grown. Illustratively, in some embodiments, the recombinant protein is purified from the harvest permeate by one or more of flocculation, precipitation, centrifugation, depth filtration, affinity chromatography, size exclusion chromatography, ion exchange chromatography, mixed mode anion exchange chromatography, hydrophobic interaction chromatography, or hydroxyapatite chromatography.

A capture unit operation may include capture chromatography that makes use of resins and/or membranes containing agents that will bind to the recombinant protein of interest, for example, affinity chromatography, size exclusion chromatography, ion exchange chromatography, hydrophobic interaction chromatography (HIC), immobilized metal affinity chromatography (IMAC), and the like. Such chromatographic materials are known in the art and are commercially available. For instance, if the recombinant protein is an antibody or contains components derived from an antibody (e.g., a Fc domain), affinity chromatography using ligands such as Protein A, Protein G, Protein A/G, or Protein L may be employed as a capture chromatography unit operation to further purify the recombinant protein. In other embodiments, the recombinant protein of interest may comprise a polyhistidine tag at its amino or carboxyl terminus and subsequently purified using IMAC. Recombinant proteins can be engineered to include other purification tags, such as a FLAG® tag or c-myc epitope and subsequently purified by affinity chromatography using a specific antibody directed to such tag or epitope.

Additional unit operations to inactivate, reduce, and/or eliminate viral contaminants may include filtration processes and/or adjusting solution conditions. One method for achieving viral inactivation is incubation at low pH (e.g., pH<4). A low pH viral inactivation operation can be followed with a neutralization unit operation that readjusts the virus inactivated solution to a pH more compatible with the requirements of the subsequent unit operations. A low pH viral inactivation operation may also be followed by filtration, such as depth filtration, to remove any resulting turbidity or precipitation. Adjusting the temperature or chemical composition (e.g., use of detergents) can also be used to achieve viral inactivation. Viral filtration can be performed using micro- or nano-filters, such as those available from Asahi Kasci (Plavona®) and EDM Milliporc (VPro®).

A polishing unit operation may make use of various chromatographic methods for the purification of the protein of interest and clearance of contaminants and impurities. The polish chromatography unit operation may make use of resins and/or membranes containing agents that can be used in either a “flow-through mode,” in which the protein of interest is contained in the eluent and the contaminants and impurities are bound to the chromatographic medium, or “bind and elute mode,” in which the protein of interest is bound to the chromatographic medium and eluted after the contaminants and impurities have flowed through or been washed off the chromatographic medium. Examples of such polish chromatography methods include, but are not limited to, ion exchange chromatography (IEX), such as anion exchange chromatography (AEX) and cation exchange chromatography (CEX); hydrophobic interaction chromatography (HIC); mixed modal or multimodal chromatography (MM), hydroxyapatite chromatography (HA); reverse phase chromatography, and size-exclusion chromatography (e.g. gel filtration).

Purified recombinant protein may be formulated, i.e., buffer exchanged, sterilized, bulk-packaged, and/or packaged for a final user. Illustratively, product concentration and buffer exchange of the recombinant protein of interest into a desired formulation buffer for bulk storage of the drug substance or drug product can be accomplished by ultrafiltration and diafiltration. Suitable formulations for pharmaceutical compositions include those described in Remington's Pharmaceutical Sciences, 18th ed. 1995, Mack Publishing Company, Easton, PA.

### Recombinant Proteins

Any type of recombinant protein comprising an AEP cleavage site, including proteins containing single polypeptide chains or multiple polypeptide chains, can be harvested according to the methods of the present disclosure. Recombinant proteins of the present disclosure include, but are not limited to, secreted proteins, non-secreted proteins, intracellular proteins, or membrane-bound proteins. Illustratively, recombinant proteins can include, but are not limited to, cytokines, growth factors, hormones, muteins, fusion proteins, antibodies, antibody fragments, peptibodies, T-cell engaging molecules, and multi-specific antigen binding proteins. In some embodiments, the recombinant protein is a fusion protein.

In other embodiments, the recombinant protein to be harvested according to a method of the present disclosure is an antigen-binding protein. Antigen-binding proteins include, but are not limited to, antibodies, peptibodies, antibody derivatives, antibody analogs, fusion proteins (including, e.g., single-chain variable fragments (scFvs), double-chain (divalent) scFvs, and IgGscFv (see, e.g., Orcutt et al., 2010, Protein Eng Des Sel 23:221-228)), hetero-IgGs (see, e.g., Liu et al., 2015, J Biol Chem 290:7535-7562), muteins, and XmAb® (Xencor, Inc., Monrovia, CA). Additional antigen-binding proteins include, but are not limited to, bispecific T cell engager (BITE®) molecules, bispecific T cell engagers having extensions, such as, e.g., half-life extensions, such as, e.g., HLE BiTE molecules, Heterolg BITE molecules, and others, chimeric antigen receptors (CARs, CAR Ts), and T cell receptors (TCRs).

In some embodiments, the antigen-binding protein binds to one of more of the following, alone or in any combination: CD proteins including, but not limited to, CD3, CD4, CD5, CD7, CD8, CD19, CD20, CD22, CD25, CD30, CD33, CD34, CD38, CD40, CD70, CD123, CD133, CD138, CD171, and CD174, HER receptor family proteins, including, for instance, HER2, HER3, HER4, and the EGF receptor, EGFRvIII, cell adhesion molecules, for example, LFA-1, Mol, p150,95, VLA-4, ICAM-1, VCAM, and alpha v/beta 3 integrin, growth factors, including but not limited to, for example, vascular endothelial growth factor (“VEGF”); VEGFR2, growth hormone, thyroid stimulating hormone, follicle stimulating hormone, luteinizing hormone, growth hormone releasing factor, parathyroid hormone, mullerian-inhibiting substance, human macrophage inflammatory protein (MIP-1-alpha), erythropoietin (EPO), nerve growth factor, such as NGF-beta, platelet-derived growth factor (PDGF), fibroblast growth factors, including, for instance, aFGF and bFGF, epidermal growth factor (EGF), Cripto, transforming growth factors (TGF), including, among others, TGF-α and TGF-β, including TGF-β1, TGF-β2, TGF-β3, TGF-β4, or TGF-β5, insulin-like growth factors-I and -II (IGF-I and IGF-II), des(1-3)-IGF-I (brain IGF-I), and osteoinductive factors, insulins and insulin-related proteins, including, but not limited to, insulin, insulin A-chain, insulin B-chain, proinsulin, and insulin-like growth factor binding proteins; (coagulation and coagulation-related proteins, such as, among others, factor VIII, tissue factor, von Willebrand factor, protein C, alpha-1-antitrypsin, plasminogen activators, such as urokinase and tissue plasminogen activator (“t-PA”), bombazine, thrombin, thrombopoietin, and thrombopoietin receptor, colony stimulating factors (CSFs), including the following, among others, M-CSF, GM-CSF, and G-CSF, other blood and serum proteins, including but not limited to albumin, IgE, and blood group antigens, receptors and receptor-associated proteins, including, for example, flk2/flt3 receptor, obesity (OB) receptor, growth hormone receptors, and T-cell receptors; neurotrophic factors, including but not limited to, bone-derived neurotrophic factor (BDNF) and neurotrophin-3, -4, -5, or -6 (NT-3, NT-4, NT-5, or NT-6); relaxin A-chain, relaxin B-chain, and prorelaxin, interferons, including for example, interferon-alpha, -beta, and -gamma, interleukins (ILs), e.g., IL-1 to IL-10, IL-12, IL-15, IL-17, IL-23, IL-12/IL-23, IL-2Ra, ILI-R1, IL-6 receptor, IL-4 receptor and/or IL-13 to the receptor, IL-13RA2, or IL-17 receptor, IL-1RAP; viral antigens, including but not limited to, an AIDS envelope viral antigen, lipoproteins, calcitonin, glucagon, atrial natriuretic factor, lung surfactant, tumor necrosis factor-alpha and -beta, enkephalinase, BCMA, IgKappa, ROR-1, ERBB2, mesothelin, RANTES (regulated on activation normally T-cell expressed and secreted), mouse gonadotropin-associated peptide, DNase, FR-alpha, inhibin, and activin, integrin, protein A or D, rheumatoid factors, immunotoxins, bone morphogenetic protein (BMP), superoxide dismutase, surface membrane proteins, decay accelerating factor (DAF), AIDS envelope, transport proteins, homing receptors, MIC (MIC-a, MIC-B), ULBP 1-6, EPCAM, addressins, regulatory proteins, immunoadhesins, antigen-binding proteins, somatropin, CTGF, CTLA4, cotaxin-1, MUC1, CEA, c-MET, Claudin-18, GPC-3, EPHA2, FPA, LMP1, MG7, NY-ESO-1, PSCA, ganglioside GD2, ganglioside GM2, BAFF, OPGL (RANKL), myostatin, Dickkopf-1 (DKK-1), Ang2, NGF, IGF-1 receptor, hepatocyte growth factor (HGF), TRAIL-R2, c-Kit, B7RP-1, PSMA, NKG2D-1, programmed cell death protein 1 and ligand, PD1 and PDL1, mannose receptor/hCGB, hepatitis-C virus, mesothelin dsFv[PE38] conjugate, Legionella pneumophila (lly), IFN gamma, interferon gamma induced protein 10 (IP10), IFNAR, TALL-1, thymic stromal lymphopoietin (TSLP), proprotein convertase subtilisin/Kexin Type 9 (PCSK9), stem cell factors, Flt-3, calcitonin gene-related peptide (CGRP), OX40L, α4β7, platelet specific (platelet glycoprotein IIb/IIIb (PAC-1), transforming growth factor beta (TFGβ), Zona pellucida sperm-binding protein 3 (ZP-3), TWEAK, platelet derived growth factor receptor alpha (PDGFRα), sclerostin, and biologically active fragments or variants of any of the foregoing.

In other embodiments, the recombinant protein to be harvested according to a method of the present disclosure is an antibody. In some embodiments, the antibody is a human antibody.

In some embodiments, the antibody is selected from abrilumab, brazikumab, brodalumab, crizanlizumab, denosumab, eculizumab, erenumab, evolocumab, fremanezumab, meplazumab, nemolizumab, ontamalimab, panitumumab, prezalumab, ravulizumab, rilotumumab, romosozumab, satralizumab, tafolecimab, tanezumab, tezepelumab, tremelimumab, utomilumab, and volagidemab. In some embodiments, the antibody is selected from denosumab, erenumab, evolocumab, panitumumab, romosozumab, and tezepelumab. In some embodiments, the antibody is denosumab. In some embodiments, the antibody is erenumab. In some embodiments, the antibody is evolocumab. In some embodiments, the antibody is panitumumab. In some embodiments, the antibody is romosozumab. In some embodiments, the antibody is tezepelumab.

In some embodiments, the antibody is an IgG2 antibody. In some embodiments, the antibody is a human IgG2 antibody.

In some embodiments, the recombinant protein to be harvested according to a method of the present disclosure comprises an IgG2 heavy chain constant region. In some embodiments, the recombinant protein comprises a wild-type IgG2 heavy chain constant region. In some embodiments, the recombinant protein comprises an engineered IgG2 heavy chain constant region.

In some embodiments, the recombinant protein comprises a CH1 domain of an IgG2 heavy chain constant region. In some embodiments, the recombinant protein comprises a N192/F193 sequence motif in a CH1 domain of a heavy chain constant region, according to EU numbering.

### Monitoring AEP Quantity or Activity or LMW Species

Some embodiments of certain methods of the present disclosure involve monitoring AEP quantity or activity and/or monitoring a quantity of LMW species of a desired recombinant protein.

In some embodiments, AEP quantity is measured using mass spectrometry or an immunoassay. In some embodiments, AEP quantity is measured using liquid chromatography-mass spectrometry (LC-MS). In some embodiments, AEP quantity is measured using nano-scale liquid chromatography-mass spectrometry (nano LC-MS).

In some embodiments, AEP activity is measured using a cleavage assay. In some embodiments in which the biomanufacturing process utilizes CHO cells, AEP peptides containing the activating auto-cleavage sites N³²⁵, D³⁰⁵ and E³¹¹ (LMSTN³²⁵DLK (SEQ ID NO: 1) and LD³⁰⁵LTPSPE³¹IVPLTILK (SEQ ID NO: 2)) may be used as indicators of cleavage and activation. AEP peptides containing alternative auto-cleavage sites may be used to monitor activation for biomanufacturing processes utilizing alternative mammalian host cells.

In some embodiments, AEP proenzyme quantity is measured using mass spectrometry or an immunoassay. In some embodiments, AEP proenzyme quantity is measured using liquid chromatography-mass spectrometry (LC-MS). In some embodiments, AEP proenzyme quantity is measured using nano-scale liquid chromatography-mass spectrometry (nano LC-MS). In some embodiments in which the biomanufacturing process utilizes CHO cells, AEP peptides containing the activating auto-cleavage sites N³²⁵, D³⁰⁵ and E³¹¹ (LMSTN³²⁵DLK and LD³⁰⁵LTSPE³¹¹VPLTILK) are used as indicators of cleavage and activation from AEP proenzyme to mature AEP.

In some embodiments, the quantity of LMW species is measured using mass spectrometry or an immunoassay, e.g., a bioassay and/or potency assay. In some embodiments, the quantity of LMW species is measured using a reduced capillary electrophoresis-sodium dodecyl sulfate method. In some embodiments, the quantity of LMW species is measured using a reduced reversed-phase (RP) chromatography method. In some embodiments, the quantity of LMW species is measured using an ultra-high-pressure liquid chromatography method.

### EXAMPLES

In order that the present disclosure may be more fully understood, the following examples are set forth. It should be understood that these examples are for illustrative purposes only and are not to be construed as limiting this disclosure in any manner.

### Example 1. AEP Induced Fragmentation of an IgG2 Antibody

A relatively high level of fragmentation was detected by rCE-SDS during the development of a high-yield biomanufacturing process in CHO cells for an IgG2 antibody (mAb1). Applicant determined that fragmentation was occurring at a N192/F193 site (in EU numbering) in mAb1; this site is a conservative motif in the CH1 domain of the IgG2 heavy chain. The human IgG2 CH1 domain is provided below as SEQ ID NO: 4 with relevant residues underlined.

(SEQ ID NO: 4)
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTV

LC-MS proteomics identified the presence of enzyme asparaginyl endopeptidase (AEP), also known as legumain, in biomanufacturing process pools. AEP is a lysosomal cysteine endopeptidase, which exhibits fragmentation activity at mildly acidic pH (2.5<pH<4.5) and negligible activity at pH 5 and above (Zhao, L. et al., Cell Res 2014, 24 (3), 344-358). AEP is originally present during cell culture in a proenzyme form, which converts to mature, active form by auto-cleavage of terminal domains at low pH. The studies described below confirmed the presence of AEP in the high yield biomanufacturing process and revealed that AEP was cleaving mAb1 primarily at low pH during an acid precipitation operation of antibody harvest from the bioreactor. Acid precipitation is a common step used to precipitate cells, DNA, and other cell debris before downstream purification, while maintaining an antibody in a soluble form. LC-MS analysis also showed that the AEP proenzyme form was converted to a mature, active form by auto-cleavage of terminal portions at low pH during acid precipitation. Because AEP enzymatic activity is higher at low pH, raising the AP target pH from 4.6 to 5.1 led to a decrease in fragmentation, as demonstrated by a reduction in LMW (as measured by a rCE-SDS assay) in drug substance from approximately 5% to approximately 0.3%. Taken together, this work identified AEP cleavage as a cause of fragmentation during low pH acid precipitation operations for recombinant proteins comprising an AEP cleavage site, such as IgG2 antibodies, and provided a mechanistic-based understanding and mitigation strategy. Notably, AEP's impact on IgG2 antibodies has not previously been reported, likely due to the relatively low levels of AEP generated by lower yield processes or due to the absence of acid precipitation operations during cell culture harvest.

### Methods

In-process samples of mAb1 and final drug substance (DS) of mAb1 samples were collected at different stages from upstream to downstream process of three pilot-scale (2 kL bioreactor) lots: PSL1, PSL2, and PSL3. A nano LC-MS based impuriomics approach was used to identify and quantify AEP levels relative to the mAb1 product for all samples. Both the mature and proenzyme forms of AEP were measured and correlated with mAb1 fragmentation levels at different stages of the process. For further confirmation, commercially available AEP was spiked into mAb1 reference standard (RS) produced by a lower-yield biomanufacturing process, and the site and level of fragmentation were assessed and correlated with the amount of spiked AEP.

### Sample Preparation

500 μg of each sample were taken and placed into 10K MWCO Amicon centrifugal filter tubes. Yeast standard proteins glucose-6-phosphate dehydrogenase (G6PD) and inorganic pyrophosphatase (IPYR) were spiked into each sample at 200 and 100 ng/mg, respectively, and all samples were spun for 30 minutes at 14,000 rpm using a Beckman Coulter microfuge 18 centrifuge. 400 μL of denaturing buffer (6M Guanidine-HCl/pH 7.5/2 mM EDTA/20 mM Methionine/250 mM Tris) and 6 μL 0.5 M 1,4-Dithiothreitol (DTT) were added to the samples, which were then incubated at 37° C. for 30 minutes. Samples were then alkylated in dark conditions for 30 minutes by adding 14 μL of 0.5M iodoacetic acid (IAA). Alkylation was quenched by adding 8 μL 0.5M DTT. After quenching, the denaturing buffer was spun through the 10K MWCO Amicon centrifugal filter at 14,000 rpm for 30 minutes. The flow-through was discarded and 400 μL 0.1M TRIS at pH 7.5 were added before spinning for another 30 minutes at 14,000 rpm. This step was repeated one more time. 25 μL of trypsin solution aliquots (1 μg/mL) were then added to the samples, followed by the addition of 50 μl 0.1M TrisHCl at pH 7.5. After the samples were gently mixed, they were placed in an incubator at 37° C. for overnight digestion (18-20 hours). 100 μL 7.5 M Guanidine-HCl at pH 5.0 was added into samples, which were then gently shaken for 30 minutes. All samples were spun for 45 minutes at 14,000 rpm using a Beckman Coulter microfuge 18 centrifuge to collect flow-through (about 200 μl) for LC/MS analysis.

### LC

A nanoLC system (Easy-nLC 1000, Thermo Fisher Scientific) was used to separate the tryptic peptides that were collected after sample preparation. An analytical column (75 μm×250 mm, EASY-Spray™ HPLC from Thermo Scientific) with a particle size of 2 μm and a trap column (75 μm×20 mm, Acclaim™ PepMap™ 100 C18 HPLC Column from Thermo Fisher Scientific) were used, with the trap column in front of the analytical column. The peptide digests were first loaded into the trap column and then eluted from there to the analytical column for further separation. Mobile phase A was 0.1% formic acid/99.9% water and mobile phase B was 0.1% formic acid/99.9% acetonitrile. The flow rate was 300 nL/min, with the column temperature set at 40° C. A gradient of 3% to 25% B over 80 minutes was used.

### MS

A Q Exactive mass spectrometer (Thermo Scientific) was coupled with the nanoLC system for the LC-MS analysis. The nano flow source conditions were set up as follows: spray voltage=1.8 KV, transfer capillary temperature=180 C°, S lens=50 V. The full MS scans were acquired using the profile data mode with resolution of 70,000, AGC target of 3E6, Maxim injection time of 120 ms, and m/z scan range from 350-1800. The MS/MS scans were acquired using the centroid data with resolution of 17,500, AGC target of 1E5, and a maximum injection time of 200 ms. The top ten most abundant peptide ions were selected for MS/MS with dynamic exclusion for 30 seconds.

### Data Processing

A MassAnalyzer 4.10 was used to process the MS data and generate a MGF file for Mascot database search. The MS noise level was set at 3,000 with a minimal signal to noise ratio of 3.

### Mascot Database Search for HCP Identification

After the searchable file (MGF file) was generated by MassAnalyzer, it was used for Mascot database search for Host Cell Protein (HCP) identification. The search parameters used are listed below: Peptide Tolerance: 15 ppm, MS/MS Tolerance: 0.02 Da or less, Primary Digest Reagent: Trypsin; Secondary Digest Reagent: None; Missed Cleavages:0; Fixed Modifier Reagents: Carbamidomethyl C; Database UniProt CHO 2013.

### HCP Quantification

MassAnalyzer used the average peak area of the top three most abundant peptides to represent the abundance or level of the protein from which the top 3 peptides came from. The HCP level was calculated by the following formula:

HCP level (ng/mg)=the average peak area of the top 3 peptide of (HCP×HCP molecular weight)/(average peak area of the top 3 peptide of Antibody×Antibody molecular weight).

In addition to the antibody, spiked yeast standard proteins glucose-6-phosphate dehydrogenase (G6PD) and inorganic pyrophosphatase (IPYR) were used for relative quantitation of AEP and other host cell proteins.

### Results

AEP Characterization to Understand mAb1 Fragmentation

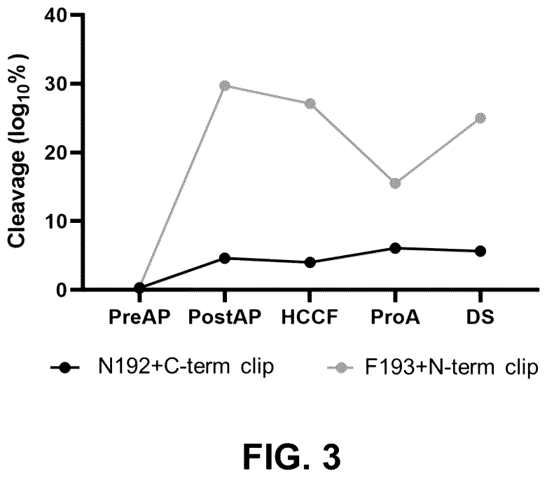

A biomanufacturing process for mAb1 was designed to achieve a high CHO cell density during production cell culture in order to increase titer. However, drug substance and in-process sample material showed increased fragmentation at the N192/F193 site (in EU numbering) of the heavy chain (FIG. 3).

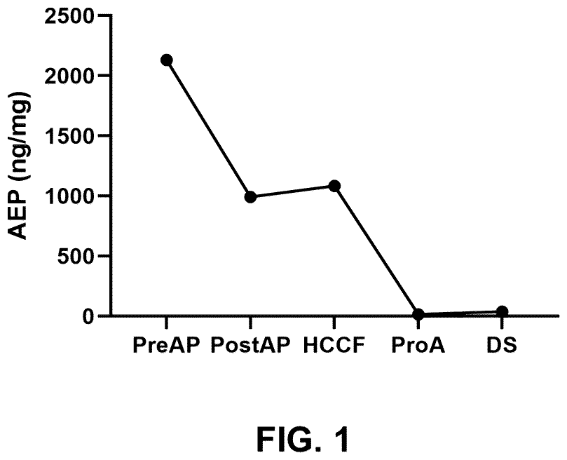

In-process samples from the pilot-scale run PSL1, which employed a target pH of 4.6 during acid precipitation and exhibited high levels of fragmentation, were used for AEP characterization. Nanoflow LC-MS/MS proteomics analysis was performed on in-process samples to understand the cause of fragmentation. AEP was detected at elevated levels, particularly in the upstream samples, which correlated with the observation that fragmentation occurs primarily in the upstream process (FIG. 1). The levels of AEP in the upstream and downstream materials showed that AEP was present in higher abundance (about 30-100 fold higher) before the downstream purification process, and most AEP was then cleared by a Protein A affinity purification step. Additionally, levels of fragmentation were relatively constant for samples collected over the downstream process, and further storage for a prolonged period of time in a pH 5.2 formulation buffer did not increase fragmentation.

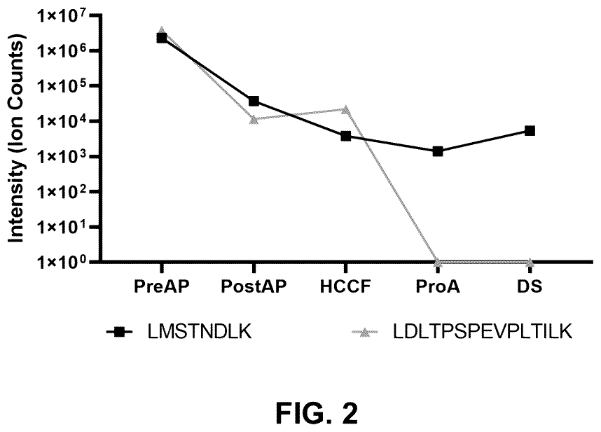

Further characterization of AEP demonstrated that its activation from a proenzyme form to a mature form occurred during acid precipitation, where the fragmentation mainly occurred. CHO AEP peptides containing the activating auto-cleavage sites N³²⁵, D³⁰⁵, and E³¹¹ (LMSTN³²⁵DLK (SEQ ID NO: 1) and LD³⁰⁵LTPSPE³¹¹VPLTILK (SEQ ID NO: 2)) were used as indicators of cleavage and activation. The intact peptides (LMSTNDLK (SEQ ID NO: 1) and LDLTSPEVPLTILK (SEQ ID NO: 2)) reflected the proenzyme form of CHO AEP. After acid precipitation, the intensities of these peptides decreased by about 100-fold, indicating that AEP was cleaved into its mature and active form at N³²⁵, D³⁰⁵, and E³¹¹ (FIG. 2), although the total AEP level remained the same before and after the acid precipitation treatment. Since AEP peptides containing the auto-cleavage sites D²⁵ and D²⁸ were not detected, they were not used as indicators of the AEP proenzyme.

To summarize, characterization of AEP for in-process samples of mAb1 was performed by nano LC-MS to understand the cause of mAb1 fragmentation. The results showed high levels of AEP in the upstream process and low levels in the downstream process. AEP levels correlated well with the observation that most of the fragmentation came from the acid precipitation step performed at pH 4.6 in the upstream process. Four parameters appeared to impact fragmentation of the IgG2 antibody mAb1: hold time; AEP concentration relative to mAb1 concentration; solution pH; and the concentration of the mature, active form of AEP after the proenzyme form was auto-cleaved.

Spiked AEP Induced Fragmentation in mAb1 Reference Standard

To further confirm that AEP was responsible for mAb1 fragmentation, 400 ng AEP per mg of antibody was spiked into a mAb1 reference standard, followed by incubation at 37° C. overnight. The spiking generated about 2.5% fragmentation at pH 4.6 while only ˜0.5% fragmentation was generated at pH 7.0 (Table 1). The level of fragmentation generated by spiked AEP (2.5%, 400 ng/mg AEP, Table 1) agreed qualitatively with what was observed for the process (4%, 1085 ng/mg AEP in HCCF sample, Table 1).

TABLE 1
Summary of characterization of AEP in mAb1 in-process samples
of lot PSL1 using a pH 4.6 AP step. AEP levels in ng/mg
and fragmentation percentages are shown in the table. LC-MS
was used for AEP and fragmentation quantification.

| | | AEP | | | |
|---|---|---|---|---|---|
| | AEP | (spiked and | | | Sam- |
| | (measured, | waited for 18 | N192 + C | F193 + N | ple |
| | ng/mg) | hours, ng/mg) | term clip | term clip | pH |
| PreAP | 2132 | 0 | 0.28% | 0.43% | 7.0 |
| PostAP | 993 | 0 | 4.63% | 29.73% | N/A |
| HCCF | 1085 | 0 | 4.02% | 27.13% | N/A |
| ProA | 15 | 0 | 6.07% | 15.50% | 3.70 |
| DS | 39 | 0 | 5.65% | 25.00% | 4.10 |
| Reference | 176 | 400 | 2.50% | 2.50% | 4.60 |
| Standard | | | | | |
| pH 4.6 | | | | | |
| Reference | 23 | 400 | 0.50% | 0.59% | 7.00 |
| Standard | | | | | |
| pH 7.0 | | | | | |
| Reference | | 0 | 0.1* | | |

### Characterization of AEP for PSL2 and PSL3 for Assessment of a Fragmentation Control Strategy

Two lots of mAb1 material were made using different pHs for the acid precipitation operation to evaluate the impact of acid precipitation pH on AEP induced fragmentation while still maintaining high yield of harvest and downstream purification: AP pH 4.9 for lot PSL2 and AP pH 5.1 for lot PSL3. High levels of AEP in upstream samples were detected in both lots (Table 2). However, PSL3 had very low levels of fragmentation, similar to that of the reference standard (as measured by rCE-SDS). The results demonstrated that increasing the pH used for the acid precipitation step to pH 5.1 for PSL3 reduced the level of AEP induced fragmentation by inhibiting both AEP activation through auto-cleavage and its activity on mAb1 since AEP enzymatic activity at pH 5.1 was significantly suppressed.

Characterization of AEP for PSL2 and PSL3 development lots showed that PSL3 had higher levels of total host cell protein (HCP) and AEP (attributed to cell culture variability), but had a lower clipping level than PSL2 (Table 2). Adjusting the acid precipitation pH from 4.6 in PSL1 to pH 5.1 in PSL3 resulted in a lower level of fragmentation. This indicated that adjustment of pH to 5.1 during AP was very effective in decreasing the clipping down to a level of RS (0.3%), despite the observation that PSL3 had a higher level of AEP. No significant differences in downstream process performance were observed in terms of AEP removal, and the clearance rates were similar at the Protein A affinity chromatography step for both PSL2 and PSL3 (Table 2). Although residual AEP levels were higher in PSL3 relative to PSL2 at the Protein A and DS steps, the level of fragmentation observed at DS was low and comparable to RS material. Levels of AEP in DS of PSL2 and PSL3 were also lower than in PSL1 and close to that of RS, presumably due to extra purification at a CEX step after Protein A (Table 2). These results demonstrate that an increase of pH to 5.1 during acid precipitation can be an effective fragmentation control strategy without other process modifications. Acid precipitation at pH 5.1 was still effective for removal of cell, DNA, and other cellular debris (data not shown).

TABLE 2
AEP levels (ng/mg) and clipping percentages (%) for in-process samples
for mAb1 PSL1 relative to mAb1 PSL2 and PSL3, measured by nano LC-MS.
Upstream

| | Total | | | | | | |
|---|---|---|---|---|---|---|---|
| | HCP | AEP | AEP | pH at | Clipping | AEP | Clipping |
| Process | (HCCF) | PreAP | PostAP | AP | at AP | HCCF | at HCCF |
| PSL1 | 250000 | 2132 | 993 | 4.6 | 2.4% | 1085 | 4.9% |
| PSL2 | 230430 | 2502 | 2759 | 4.9 | 0.5% | 1631 | 0.7% |
| PSL3 | 454996 | N/A | N/A | 5.1 | 0.4% | 4734 | 0.4% |

Downstream

| | | Clipping at | | | Clipping at |
|---|---|---|---|---|---|
| Process | AEP ProA | ProA | AEP CEX | AEP DS | DS |
| PSL1 | 15 | 5.0% | N/A | 39 | 5.0% |
| PSL2 | 6.6 | 0.7% | 3.4 | 0.3 | 0.9% |
| PSL3 | 17.4 | 0.3% | 3 | 1.4 | 0.3% |

Assessment of Impact of Residual AEP on DS Stability of mAb1 PSL3

Since residual AEP was still carried into DS, the impact of AEP on mAb1 drug substance (DS) stability was assessed using DS from PSL3. The observed fragmentation level in stressed PSL3 DS was similar to that in a stressed lot of reference standard (RS) material (Table 3), indicating that residual AEP had no significant impact on DS stability (for LMW by rCE-SDS). The aggregation rate in a SE-HPLC assay was also typical, suggesting that acid precipitation at pH 5.1 was still effective for removal of cell, DNA, and other cellular debris.

TABLE 3
mAb1 stability study data summary: DS
from the PSL3 lot vs a comparator lot

| | | | | | Main | |
|---|---|---|---|---|---|---|
| 40° C. | Weeks | Aggregate | Oligomer | HMW | Peak | LMW |
| PSL3 Lot | 0 | 0 | 1.1 | 1.1 | 98.8 | 0.1 |
| DS | 1 | 0.2 | 1.7 | 1.9 | 97.9 | 0.2 |
| | 2 | 0.4 | 2 | 2.5 | 97.2 | 0.3 |
| | 4 | 0.8 | 2.6 | 3.4 | 96.0 | 0.6 |
| Comparator | 0 | 1.1 | 1.1 | 1.1 | 98.9 | n.d. |
| Lot DS | 1 | n.d. | n.d. | n.d. | n.d. | n.d |
| | 2 | 0.9 | 2.2 | 3.1 | 96.6 | 0.3 |
| | 4 | 1.9 | 2.8 | 4.7 | 94.6 | 0.5 |

All documents, or portions of documents, cited in this application, including but not limited to patents, patent applications, articles, books, and treatises, are hereby expressly incorporated by reference. What is described in an embodiment of the disclosure can be combined with one or more other embodiments of the disclosure unless context clearly indicates otherwise.

The disclosed subject matter is not intended to be limited in scope by the specific embodiments described herein, which are instead intended as non-limiting illustrations of individual aspects of the disclosure. Functionally equivalent methods and components are within the scope of the disclosure. Indeed, various modifications of the disclosed subject matter, in addition to those shown and described herein, will be apparent to those skilled in the art from the foregoing description and accompanying drawing(s). Such modifications are intended to fall within the scope of the disclosed subject matter.

The descriptions of the various embodiments and/or examples of the disclosed subject matter have been presented for purposes of illustration, but are not intended to be exhaustive or limiting in any way. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, and/or to enable others of ordinary skill in the art to understand the disclosed subject matter.

What is claimed is:

1. A method for harvesting a recombinant protein during a biomanufacturing process comprising:
establishing a cell culture by inoculating a bioreactor with mammalian cells expressing the recombinant protein;
maintaining the cell culture during a growth phase and a production phase, wherein an exogenous protease inhibitor is optionally admixed with the cell culture during the growth phase and/or during or after the production phase;
cooling the cell culture, wherein a dissolved oxygen level of about 64 mmHg to about 128 mmHg is optionally maintained in the cell culture during the cooling;
admixing an acidic solution with the cell culture to obtain an acidified cell culture with a pH of about 4.6 to about 5.3; and
incubating the acidified cell culture to induce agglomeration of one or more cell culture impurities,
wherein the recombinant protein comprises an asparaginyl endopeptidase (AEP) cleavage site.

2. The method of claim 1, wherein an exogenous protease inhibitor is admixed with the cell culture during the growth phase, the production phase, and/or the cooling phase.

3. The method of claim 2, wherein the exogenous protease inhibitor is an AEP inhibitor.

4. The method of claim 1, wherein a dissolved oxygen level of about 64 mmHg to about 128 mmHg is maintained in the cell culture during the cooling.

5. The method of claim 1, wherein the cell culture is cooled to about 4° C. to about 15° C.

6. The method of claim 1, wherein the acidic solution comprises an acid selected from acetic acid, trichloroacetic acid, formic acid, phosphoric acid, sulfuric acid, citric acid, caprylic acid, and combinations of any of the foregoing.

7. The method of claim 1, comprising incubating the acidified cell culture for at least about 60 minutes.

8. The method of claim 1, further comprising separating the one or more cell culture impurities from the acidified cell culture to obtain a clarified cell culture by continuous solid discharge centrifugation, disk stack centfiguation, and/or depth filtration.

9. The method of claim 1, wherein the pH of the acidified cell culture is about 4.9 to about 5.2.

10. The method of claim 1, further comprising:
measuring AEP quantity or activity prior to the admixing; and/or
measuring AEP proenzyme quantity prior to the admixing.

11. The method of claim 1, wherein the recombinant protein comprises a N192/F193 sequence motif in a CH1 domain of a heavy chain constant region, according to EU numbering.

12. The method of claim 1, wherein the recombinant protein is an IgG2 antibody.

13. The method of claim 1, wherein the biomanufacturing process comprises a perfusion cell culture process, a concentrated fed batch cell culture process, or an intensified cell culture process.

14. A method for inhibiting cleavage of a recombinant protein comprising an asparaginyl endopeptidase (AEP) cleavage site during a biomanufacturing process comprising an acid precipitation, the method comprising:
measuring a quantity or activity of AEP and/or a quantity of one or more fragments of the recombinant protein in a sample isolated from a cell culture, harvest, or purification operation of the biomanufacturing process; and
adjusting the acid precipitation pH to about 4.6 to about 5.3 if the quantity or activity of AEP and/or the quantity of the one or more fragments is above a threshold.

15. The method of claim 14, wherein cleavage of the recombinant protein is inhibited relative to a recombinant protein produced by an alternative biomanufacturing process using an acid precipitation pH of less than about 4.6.

16. The method of claim 14, wherein the sample is isolated from a cell culture or harvest operation.

17. The method of claim 14, wherein:
the AEP quantity is measured using mass spectrometry or an immunoassay; or
the AEP activity is measured using a cleavage assay.

18. The method of claim 14, further comprising:
adjusting a dissolved oxygen level in the cell culture to about 64 mmHg to about 128 mmHg during a cell culture cooling operation of the biomanufacturing process if the quantity or activity of AEP is above the threshold; and/or
admixing an exogenous protease inhibitor with a cell culture during a growth phase and/or a production phase of the biomanufacturing process if the quantity or activity of AEP is above the threshold.

19. The method of claim 14, wherein:
the threshold for AEP quantity is greater than about 1000 ng AEP per 1 mg of harvest cell culture fluid (HCCF); and/or
the threshold for AEP activity is greater than about 3% clipping of the recombinant protein at the AEP cleavage site; and/or
the threshold is at least about 1% w/w of the recombinant protein is the one or more fragments.

20. The method of claim 14, comprising adjusting the acid precipitation pH to about 4.9 to about 5.2 if the quantity or activity of AEP and/or the quantity of the one or more fragments is above the threshold.

21. The method of claim 14, wherein the recombinant protein comprises a N192/F193 sequence motif in a CH1 domain of a heavy chain constant region, according to EU numbering.

22. The method of claim 14, wherein the recombinant protein is an IgG2 antibody.
